SEQUENCE LISTING (10 sequences)

SEQ ID NO: 1 - DNA, 20 bp, Artificial Sequence
  Primer sequene for human assay
gtaacccgttgaaccccatt

SEQ ID NO: 2 - DNA, 20 bp, Artificial Sequence
  Primer sequene for human assay
ccatccaatcggtagtagcg

SEQ ID NO: 3 - DNA, 23 bp, Artificial Sequence
  Primer sequene for human assay
accattgtcatatacccggttca

SEQ ID NO: 4 - DNA, 23 bp, Artificial Sequence
  Primer sequene for human assay
caatagcccaagtagccaatcat

SEQ ID NO: 5 - DNA, 22 bp, Artificial Sequence
  Primer sequene for human assay
tcaagccagatcagcaattctc

SEQ ID NO: 6 - DNA, 21 bp, Artificial Sequence
  Primer sequene for human assay
agccagtttcatctccacatg

SEQ ID NO: 7 - DNA, 21 bp, Artificial Sequence
  Primer sequene for human assay
atccagaacttcgaccttccg

SEQ ID NO: 8 - DNA, 19 bp, Artificial Sequence
  Primer sequene for human assay
acggcattgagcgtgtagg

SEQ ID NO: 9 - DNA, 21 bp, Artificial Sequence
  Primer sequene for human assay
gtggagcgatttagccaagaa

SEQ ID NO: 10 - DNA, 21 bp, Artificial Sequence
  Primer sequene for human assay
cacaaggcatcgtttcaatgg

(19) United States
(12) Patent Application Publication
(10) Pub. No.: US 2025/0275946 A1
(43) Pub. Date: Sep. 4, 2025

(54) COMBINATION THERAPY FOR THE TREATMENT OF CANCER

(71) Applicant: UNM RAINFOREST INNOVATIONS, Albuquerque, NM (US)

(72) Inventor: Xiang XUE, Albuquerque, NM (US)

(21) Appl. No.: 18/269,854

(22) PCT Filed: Jan. 25, 2022
(86) PCT No.: PCT/US2022/013676

§ 371 Date: Jun. 27, 2023

Related U.S. Application Data
(60) Provisional application No. 63/144,788, filed on Feb. 2, 2021.
(60) Provisional application No. 63/232,996, filed on Aug. 13, 2021.

(51) Int. Cl. A61K 31/4196; A61K 31/16; A61K 45/06; A61N 5/00; A61P 35/00

(52) U.S. Cl. CPC A61K 31/4196; A61K 31/16; A61K 45/06; A61N 5/00; A61P 35/00

(57) ABSTRACT

The present invention is directed to the unexpected discovery that iron chelators and anticancer agents, such as checkpoint kinase 1 (CHK1) inhibitors, ATR inhibitors and DNA damaging agents, and/or radiotherapy when combined in effective amounts, exhibit a synergistic effect in the inhibition and treatment of cancer. Accordingly, the present invention is directed to methods for the treatment of cancer which combine effective amounts of an iron chelator and a Chk1 inhibitor, a ATR inhibitor, a DNA damaging agent and/or radiotherapy. Optionally, an additional anticancer agent may be used in the treatment of cancer. In alternative embodiments, the present invention is directed to pharmaceutical compositions which are used in the treatment of cancer and comprise an effective amount of at least one iron chelator, and at least one or more of a Chk1 inhibitor, ATR inhibitor and/or DNA damaging agent, optionally in combination with one of more additional anticancer agent as described herein in combination with a pharmaceutically acceptable carrier, additive or excipient.

### RELATED APPLICATIONS

This application claims the benefit of priority of U.S. provisional application Ser. No. 63/144,788, filed Feb. 2, 2021 and Ser. No. 63/232,996, filed Aug. 13, 2021 of identical title, both of said applications being incorporated by reference in their entirety herein.

### GRANT SUPPORT

This invention was made with government support under grant number P20 GM130422 awarded by National Institutes of Health (NIH). The government has certain rights in the invention.

### FIELD OF THE INVENTION

The present invention is directed to the unexpected discovery that iron chelators and other anticancer therapies, including checkpoint kinase 1 (Chk1) inhibitors, ataxia telangiectasia mutated and Rad3 related kinase inhibitors (ATR inhibitors), DNA damaging agents and radiotherapy among other anticancer agents and therapy, when combined in effective amounts, exhibit a synergistic effect in the inhibition and treatment of cancer. Accordingly, the present invention is directed to methods for the treatment of cancer which combine effective amounts of an iron chelator and anticancer therapy to provide synergistic effect on cancer. In the present invention, an iron chelator is combined with an anticancer agent including a checkpoint kinase 1 (Chk1) inhibitor, an ataxia telangiectasia mutated and Rad3 related kinase inhibitor (ATR inhibitor), a DNA damaging and/or other anticancer agents and therapies such as radiotherapy in order to treat cancer in a synergistic matter. In other embodiments, the present invention is directed to pharmaceutical compositions which are used in the treatment of cancer and comprise an effective amount of at least one iron chelator in combination with one of more checkpoint kinase 1 inhibitors, ATR inhibitors, DNA damaging agents and other anticancer therapies in combination with a pharmaceutically acceptable carrier, additive or excipient.

### BACKGROUND AND OVERVIEW OF THE INVENTION

Several large epidemiological studies have shown a direct correlation between high systemic iron levels and colorectal cancer (CRC) risk, whereas iron was accumulated in both human and animal models of CRC. However, the precise mechanism leading to efficient intratumoral iron accumulation and the role of iron metabolism plays in colon carcinogenesis are still unclear. Transferrin receptor (TFRC) is known as a major iron uptake protein that delivers iron into cells. Here we found that TFRC expression was significantly increased in human and mouse colon tumors compared to adjacent normal colon tissues, and this increase was further enhanced by high iron diet. Furthermore, colon-specific deletion of TFRC led to increased intestinal epithelial cell apoptosis and susceptibility to colitis. However, TFRC disruption prolonged survival in a mouse model of colon dysplasia caused by biallelic Apc loss and decreased colon tumorigenesis in a mouse model with single allele of Apc gene loss. Mechanistically, disruption of TFRC led to a reduction of colon iron levels and subsequent decreased c-Myc-E2F1-DNA polymerase delta1 (POLD1) axis. Inhibition of POLD1, an enzyme that is critical for DNA replication and repair, led to increased DNA damage response and apoptosis, and reduced colon tumor growth. Consistently, disruption of TFRC and iron chelation also increased DNA damage response and apoptosis. Importantly, combinational inhibition of POLD1 by iron chelation and DNA repair by CHK1 inhibitors caused a synergistic effect in reducing colon tumor cell growth. In summary, our results suggest that TFRC-iron-POLD1 plays an important role in colon homeostasis and provides a novel strategy for CRC targeted therapy.

Colorectal cancer (CRC) is the third leading cause of cancer-related death in the US [1]. Understanding the mechanisms of CRC onset and progression is essential to improve treatment. Many tumor cells exhibit metabolic reprogramming such as increased glycolysis and nucleotide synthesis in metabolomics studies [2]. The metabolic differences between normal and cancer cells are being interrogated as novel therapeutic targets [3], but little has been focused on micronutrients. Decades of research by numerous investigators firmly placed micronutrient iron as a major regulator of colon tumorigenesis. Data from several epidemiological studies demonstrate a direct correlation between increased systemic/tissue iron levels and high CRC risk [4-8]. Iron is accumulated in human and animal models of CRC [9, 10]. The expression of proteins associated with iron uptake machinery divalent metal transporter 1 (DMT1) and transferrin receptor (TFRC) are up-regulated in CRC [11]. Previously, we have shown that pharmacological inhibition or deletion of apical ferrous iron importer DMT1 greatly reduced intratumoral iron uptake and colon tumorigenesis in mice [11]. However, the role of TFRC in CRC is still unknown.

TFRC, also known as TfR1 and CD71, is a transmembrane glycoprotein expressed on the cell surface. TFRC is known as the major iron uptake protein that delivers iron into cells through receptor-mediated endocytosis of diferric transferrin to maintain intracellular iron homeostasis, whereas itself is often recycled back to the plasma membrane after endocytosis through recycling endosomes [12]. In normal intestine, TFRC is absent on the microvilli of villous enterocytes both in human and rat duodenal samples [13, 14]. Instead, TFRC is located in the basolateral area of the cytoplasm, suggesting that TFRC is not acting as an iron carrier from the intestinal lumen into the cells [14, 15]. This was further validated by the fact that all intestinal epithelial cell-specific TFRC knockout mice died within 3 days after born, but iron-loading did not rescue their viability [16].

However, under anemia condition, patients showed a significant increase of TFRC expression in the villous enterocytes [15]. Moreover, many types of cancer cells including CRC have higher TFRC expression than normal cells, herein TFRC is a potential molecular target for diagnosis and treatment for cancer therapy [17]. For example, anti-human TFRC monoclonal antibody A24 impaired TFRC expression and recycling, blocked the proliferation of T-cell leukemia cell, induced apoptosis of malignant T lymphocytes from adult T-cell leukemia patients [18]. Other recombinant antibodies targeting human TFRC can also cause TFRC degradation and lethal iron deprivation in malignant B cells and xenograft models [19-22].

In the present study, we found that TFRC is required for maintaining colon tissue homeostasis. High expression of TFRC led to increased iron uptake and accumulation in CRC, whereas TFRC disruption caused iron reduction in colon tissues. Transcriptomics and proteomics analysis identified that iron restriction inhibited the iron-sulfur protein DNA polymerase delta 1 (POLD1). Downregulation of c-Myc-regulated E2F1 was responsible for POLD1 inhibition under low iron condition, whereas POLD1 reduction caused increased DNA damage response, apoptosis and repressed tumor growth. Importantly, a synergic effect was found between iron restriction and CHK1 inhibition, which provides a potential novel targeted therapy strategy for CRC.

### BRIEF DESCRIPTION OF THE INVENTION

The present invention is directed to the treatment of cancer in a patient in need comprising administering to said patient an effective amount of a combination of an iron chelator in combination with an anticancer agent (e.g. a checkpoint kinase 1 (Chk1) inhibitor, an ATR inhibitor, a DNA damaging or other anticancer agent) and/or radiotherapy to provide an unexpected synergistic inhibition of cancer. The present invention thus is directed in a first embodiment to a method of treating cancer comprising administering to a patient or subject in need an effective amount of a combination of at least one iron chelator and at least one anticancer agent (a checkpoint kinase 1 (Chk1) inhibitor, ATR inhibitor, DNA damaging agent or other anticancer agent, including mixtures thereof) and/or radiotherapy. In an alternative embodiment, the present invention is directed to pharmaceutical compositions comprising an anti-cancer effective amount of at least one iron chelator in combination with an effective amount of at least one anticancer agent (checkpoint kinase 1 (Chk1) inhibitor, ATR inhibitor, DNA damaging agent and/or another anticancer agent) further in combination with a pharmaceutically acceptable carrier, additive and/or excipient. In embodiments, the combination of an iron chelator and an anticancer agent and/or radiotherapy (radiation therapy), especially a checkpoint kinase inhibitor, ATR inhibitor and/or a DNA damaging agent or other anticancer agent, provide a synergistic cytotoxic effect on the cancer which is treated.

In embodiments, the iron chelator is a compound selected from the group consisting of deferoxamine, deferasirox, Dp44mT, dexrazoxane, ciclopirox, dexrazoxane HCl (ICRF-187), pentetate calcium trisodium hydrate, 2,3-dihydroxybenzoic acid, VLX600, L-mimosine, N-NE3TA-NCS, CAB-NE3TA, DFT (2-(3′-hydroxypyrid-2′-yl)4-methyl-delta2-thiazoline-4(S)-carboxylic acid; desferrithiocin), 4-(OH)-DADFT, 4′-(HO)-DADMDFT ((S)-2-(2,4-dihydroxyphenyl)-4,5-dihydro-4-thiazolecarboxylic acid), BDU ((S,S)-1,11-bis[5-(4-carboxy-4,5-dihydrothiazol-2-yl)-2,4-dihydroxyphenyl]-4,8-dioxaundecane), ICL6770A (4-[3,5-bis-(hydroxyphenyl)-1,2,4-triazol-1-yl]-benzoic acid), DFP (3-Hydroxy-1,2-dimethyl-4(1H)-pyridone; Deferiprone), CP94 (Diethyl hydroxypyridinone), CP502 (1,6-dimethyl-3-hydroxy-4-(1H)-pyridinone-2-carboxy-(N-methyl)-amide hydrochloride), TREN-(Me-3,2-HOPO) (N,N′,N″-tris[(3-hydroxy-1-methyl-2-oxo-1,2-didehydropyrid-4-yl)carboxamidoethyl]amine), Pr-(Me-3,2-HOPO) (3-Hydroxy-1-methyl-2-oxo-1,2-dihydro-pyridine-4-carboxylic acid propylamide), Tachpyridine (N,N′,N″-tris(2-pyridylmethyl)-cis,cis-1,3,5-triaminocyclohexane),PIH, SIH (Salicylaldehyde isonicotinoyl hydrazone), 311 (2-hydroxy-1-naphthylaldehyde isonicotinoyl hydrazone), 5-HP (5-hydroxypicolinaldehyde thiosemicarbazone), D-Exo 772SM, PCIH, INH, Deferitazole, EDTA, DTPA, Succimer, Trientine, BPS, PCTH, PCBH, PCBBH, PCAH, PCHH, FIH, Quercetin, or a pharmaceutically acceptable salt or mixture thereof. In embodiments, the checkpoint kinase 1 (Chk1) inhibitor is a compound selected from the group consisting of prexasertib, UCN-01, SRA737, AZD7762, rabusertib (LY2603618), MK-8776 (SCH 900776), CHIR-124, PF-477736, VX-803 (M4344), GDC-0575 (ARRY-575), PD0166285, CCT245737 (SRA737, PNT-737), SAR-020106, SB 218078, prexasertib, UCN-01, SRA737, AZD7762, rabusertib (LY2603618), MK-8776 (SCH 900776), CHIR-124, PF-477736, VX-803 (M4344), GDC-0575 (ARRY-575), PD0166285, SAR-020106, SB 218078, TCS2312, aminothiadiazole and aminothiadiazole conjugated cyanopyridines, CCT245737 (SRA737, PNT-737), CCT245737(S), CCT244747, GDC-0425, GNE-783, GNE-900, GO-6976, MU-380, NSC30049, PD0407824, PD-321852, V158411 or a pharmaceutically acceptable salt or mixture thereof. In embodiments, the ATR inhibitor is one or more of VE-822 (VX-970, M6620), Elimusertib (BAY1895344), VX-803, EPT-46464, AZ20, ceralasertib (AZD 6738) and VE-821, CGK 733, schisandrin B, HAMNO and Torin 2, among others. In still other embodiments the DNA damaging agent or anticancer agent is a DNA reactive agent, an antimetabolite, a topoisomerase inhibitor, and an anthracycline (e.g. doxorubicin or daunorubicin, among others), or other anticancer agent as set forth in further detail herein. In embodiments, the radiotherapy used in combination with the iron chelator is external beam radiation therapy, contact x-ray brachytherapy, brachytherapy (sealed source radiotherapy), radionuclide therapy and/or intraoperative radiotherapy.

These and other embodiments of the present invention may be readily gleaned from a review of the detailed description of the invention and the examples which are set forth herein.

### BRIEF DESCRIPTION OF THE FIGURES

FIG. 1 shows that TFRC is increased in CRC. Detection of TFRC expression in human normal and tumor colon tissues by (A) qPCR analysis, (B) immunoblotting analysis and (C) quantification, and (D) IF staining and (E) quantification. Tfrc expression in mouse normal and tumor colon tissues by (F) qPCR analysis, (G) immunoblotting blot analysis and (H) quantification. CDX2^{ERT2} Apc^{F/+} mice were fed with a 40Fe or 1000Fe iron diet. *p<0.05 and **p<0.01.

FIG. 2 shows that TFRC disruption causes colonic injury and increased susceptibility to acute colitis under low iron condition. (A) Macroscopic images, (B) immunoblotting analysis, (C) H&E staining images, (D) histological injury score, and (E) CC3 staining and (F) quantification of colons from CDX2^{EFT2} Tfrc^{F/F} and Tfrc^{F/F} mice treated with 100 mg/kg TAM for 3 days. Arrowheads indicate tissue injury. (G) Body weight change, (H) macroscopic images, (I) colon lengths, (J) H&E staining images and (K) histological scoring of colons from CDX2^{ERT2} Tfrc^{F/F} and Tfrc^{F/F} mice treated with 100 mg/kg TAM for 3 days followed with 3.5Fe and 2% DSS for 7 days. *p<0.05 and **p<0.01.

FIG. 3 shows that TFRC disruption prolongs mouse survival and reduces low-grade dysplasia caused by biallelic Apc loss. (A) Survival curve in CDX2^{ERT2} Tfrc^{F/F}Apc^{F/F} and CDX2^{ERT2} Tfrc^{+/+}Apc^{F/F} mice treated with 100 mg/kg TAM for 3 days and 1.5% DSS for 7 days. (B) qPCR analysis, (C) immunoblotting analysis, (D) macroscopic and H&E staining images, (E) histological scores, (F) IF staining and (G) quantification of colons from CDX2^{ERT2} Tfrc^{F/F}Apc^{F/F} and CDX2^{ERT2} Tfrc^{+/+}Apc^{F/F} mice treated with 100 mg/kg TAM for 3 days and euthanized 7 days later. *p<0.05 and **p<0.01. NS, not significant.

FIG. 4 shows that TFRC depletion reduces colon tumorigenesis. (A) Macroscopic images, (B) tumor number at different sizes, (C) total tumor number, (D) tumor burden, (E) qPCR analysis, (F-I) histological staining and quantification of colons from CDX2^{ERT2} Tfrc^{F/F}Apc^{F/+} and CDX2^{ERT2} Tfrc^{+/+}Apc^{F/+} mice treated with 100 mg/kg TAM for 3 days and 2 cycles of 2% DSS for 7 days with an interval of 14 days regular water. *p<0.05, **p<0.01 and ***p<0.001. NS, not significant.

FIG. 5 shows that the DNA polymerase POLD1 is regulated by iron/TNKS/Axin2/c-Myc/E2F1 axis. (A) RNA-seq analysis followed by DAVID bioinformatics analysis and KEGG pathway enrichment identified decreased expression of the nucleotide metabolic enzymes RRM2 and POLD1 after DFO treatment in colonoids. Tumor colonoids were treated with DFO (0 or 100 μM) in KGMG medium for 4 days. (B) qPCR analysis of mRNA expression of RRM2 and POLD1 in colonoids after DFO treatment. (C) Immunoblotting blot analysis in colon-derived HCT116 or SW480 cancer cells following 100 μM DFO treatment. (D) Immunoblotting analysis of proteins pulled down (PD) by Fe²⁺ or empty beads in HCT116 cells is shown. (E) Immunoblotting analysis of HCT116 cells treated with or without 100 μM DFO and/or different doses of FS. (F) Immunoblotting analysis of POLD1 expression in colons from C57BL/6 mice treated with 3.5Fe and 40Fe. Immunoblotting analysis of SW480 cells transfected with (G) E2F1, (H) c-Myc and treated with or without 100 μM DFO. Immunoblotting analysis in SW480 cells transfected with (I) siE2F1 or (J) siMYC and a scrambled control (siScr) for 24 hours. Topflash luciferase assay in SW480 cells transfected with TNKS and empty vector (EV) for 24 hours and then treated with (K) 100 μM DFO or (L) 100 μM FS for an additional 24 hours. (M) Immunoblotting analysis of SW480 cells transfected with TNKS for 24 hours and then treated with 100 μM FS treatment for overnight. Immunoblotting analysis of SW480 cells treated with (N) DFO and/or TNKS activity inhibitor XAV939, (O) FS and/or XAV939. **p<0.01 and ***p<^{0.001}.

FIG. 6 shows that TFRC depletion causes decreased iron, POLD1 and tumor growth. Immunoblotting analysis in (A) dysplastic colons from CDX2^{ERT2} Tfrc^{F/F}Apc^{F/F} and CDX2^{ERT2} Tfrc^{+/+}Apc^{F/F} mice, (B) colon tumors from CDX2^{ERT2} Tfrc^{F/F}Apc^{F/F} and CDX2^{ERT2} Tfrc^{+/+}Apc^{F/+} mice, (C) RKO cells transfected with siTFRC for 24 hours. (D) Immunoblotting analysis, (E) Ferro-Orange staining and (F) quantification, (G) MTT assay, (H) colony formation assay in MC38 cells with or without TFRC knockdown. (I) Representative tumor xenograft images, (J) tumor weight, (K) tumor iron levels and (L) immunoblotting analysis of tumor xenografts from MC38 cells with or without TFRC knockdown. *p<0.05, **p<0.01 and ***p<0.001.

FIG. 7 shows that POLD1 inhibition leads to increased DNA replication stress and impaired tumor growth. (A) Gene expression of POLD1 in colons from TCGA database. (B) Immunoblotting analysis and (C) quantification of POLD1 expression in human normal and tumor colons. (D) Immunoblotting analysis, (E) MTT assay and (F) colony formation assay in MC38 cells with or without stable POLD1 knockdown. (G) Representative tumor xenograft images, (H) tumor weight, (I) Immunoblotting analysis, (J) IF staining, quantification of (K) CC3 and (L) γH2AX staining in tumor xenografts from MC38 cells with or without POLD1 knockdown. (M) γH2AX staining and quantification of (N) dysplastic colon tissues from CDX2^{ERT2} Tfrc^{F/F}Apc^{F/F} and CDX2^{ERT2} Tfrc^{+/+}Apc^{F/F} mice and (O) colon tumor tissues from CDX2^{ERT2} Tfrc^{F/F}Apc^{F/+} and CDX2^{ERT2} Tfrc^{+/+}Apc^{F/+} mice. (P) MTT assay and (Q) Immunoblotting analysis in MC38 cells treated with 10 μM DFX and/or 10 μM 5-FU for 48 hours. (R) tumor weight for xenografts from MC38 cells treated with DFX and/or 5-FU. *p<0.05, **p<0.01 and ***p<0.001. NS, not significant.

FIG. 8 shows that iron reduction leads to DNA damage response and cell apoptosis. (A) Immunoblot analysis in colon-derived HCT116 or SW480 cancer cells following different doses of DFO treatment. (B) DNA damage marker γH2AX staining of dysplastic colon tissues from CDX2ERT2 TfrcF/FApcF/F and CDX2ERT2 Tfrc+/+ApcF/F mice and colon tumor tissues from CDX2ERT2 TfrcF/FApcF/+ and CDX2ERT2 Tfrc+/+ApcF/+ mice, from CDX2ERT2 ApcF/+ mice.

FIG. 9 shows that Iron reduction sensitizes CRC cells to inhibitors of ATR-CHK1 DNA damage signaling pathway. (A, B) MTT assay and (C, D) immunoblot analysis in SW480 treated with 10 μM iron chelator DFX and/or 100 nM CHK1 inhibitor Prexasertib and MC38 cells treated with 10 μM DFX and/or Prexasertib 20 nM for 48 hours. (E) MTT assay and (F) Immunoblot analysis in SW480 treated with 10 μM DFX and/or 1 μM ATR inhibitor VE-822 for 48 hours. **p<0.01 and ***p<0.01.

FIG. S1 shows cellular and intracellular localization of TFRC in human normal colon and tumor tissues. (A) Representative immunohistochemical staining shows epithelial and stromal localization of TFRC in normal human colon tissues. (B) Representative immunohistochemical staining shows basolateral and intracellular localization of TFRC in human colon tumors. Image credit: Human Protein Atlas. Gene expression of TFRC (C) in normal and colon tumors, (D) based on individual cancer stages from TCGA database. Protein expression of TFRC (E) in normal and colon tumors, (F) based on individual cancer stages from CPTAC database.

FIG. S2 shows the characterization of colon-specific TFRC deletion mice. (A) Body weight changes, (B) colon lengths, (C) representative images and (D) quantification of Ki67 staining of colons, (E) FITC-dextran fluorescence intensity from serum of CDX2^{ERT2}Tfrc^{F/F} and Tfrc^{F/F} mice treated with 100 mg/kg TAM for 3 days. (F) Body weight changes and (G) colon lengths of CDX2^{ERT2} Tfrc^{F/F} and Tfrc^{F/F} mice treated with 100 mg/kg TAM for 3 days and 3% DSS for 7 days. NS, not significant.

FIG. S3 shows that rapamycin didn't prolong mouse survival caused by bi-allelic Apc loss. (A) DAB-enhanced Perl's iron staining, (B) immunoblotting analysis in CDX2^{ERT2} Tfrc^{F/F}Apc^{F/F} and CDX2^{ERT2} Tfrc⁺⁺Apc^{F/F} mice treated with 100 mg/kg TAM for 3 days and sacked after 7 days of last TAM treatment. (C) Survival curve in CDX2^{ERT2}Apc^{F/F} mice treated with 100 mg/kg TAM for 3 days and 1.5% DSS for 7 days. Daily i.p. injection of 10 mg/kg rapamycin was performed at one day before DSS treatment.

FIG. S4 shows that colon-specific TFRC deletion protects mice from high iron-driven colon tumorigenesis. (A) Immunoblotting blot analysis, (B) DAB-enhanced Perl's iron staining, (C) total tumor number, (D) tumor burden, (E) tumor number at different sizes, (F) average tumor size in CDX2^{ERT2} Tfrc^{F/F}FApc^{F/+} and CDX2^{ERT2} Tfrc^{+/+}Apc^{F/+} mice treated with 100 mg/kg TAM for 3 days and 2 cycles of 2% DSS for 7 days with an interval of 14 days regular water. Mice were treated with 40Fe or 1000Fe as indicated. **p<0.01, ***p<0.001. NS, not significant.

FIG. S5 shows that POLD1 is regulated by E2F1 and binds with iron. Immunoblotting blot analysis in SW480 cells treated with or without (A) HIF-1α inhibitor PX-478, (B) HIF-2α inhibitor PX-2385 following DFO treatment. (C) Immunoblotting blot analysis in HCT116 p53+/+ and p53−/− cells with or without DFO treatment (D) Immunoblotting blot analysis of HCT116 or SW480 cells treated with different doses of DFO. (E) Immunoblotting blot analysis of RKO cells transfected with HA tagged E2F1 and treated with or without DFO for 24 hours. (F) Immunoblotting blot analysis of HEK293T cells transfected with c-Myc and treated with or without DFO for overnight. (G) Immunoblotting blot analysis of SW480 cells treated or without c-Myc inhibitor 10058-F4 for overnight. (H) Topflash assay in SW480 transfected with or without □-catenin followed with or without DFO treatment for overnight. (I) Immunoblotting blot analysis in SW480 treated with different doses of FS. (J) Immunoblotting blot analysis in HCT116 or SW480 treated with different doses of DFO. (K) Immunoblotting blot analysis in SW480 transfected with Control (siScr) or AXIN2 siRNA. (L) Immunoblotting blot analysis in SW480 treated with different doses of FS or DFO. (M) Immunoblotting blot analysis in SW480 transfected siScr or TNKS siRNA. (N) Immunoblotting blot analysis of normal and tumor colon tissues from CDX2^{ERT2} Apc^{F/+} mice fed with 40Fe or 1000Fe iron diet. ***p<0.001.

FIG. S6 shows that E2F1 is critical for POLD1 regulation in TFRC knockdown cells. (A) Immunoblot analysis of MC38 shTFRC or shEV cells. (B) Topflash assay in SW480 treated siScr or TFRC siRNA. (C) Immunoblotting blot analysis of MC38 shTFRC or shEV cells transfected with HA-E2F1. (D) Representative Ferro-Orange staining images of MC38 cell treated with vehicle control (CTRL), FS or DFO. (E) Representative crystal violet staining images for colony formation assay from MC38 cells with or without TFRC knockdown. ***p<0.001.

FIG. S7 shows that the Iron-POLD1 axis is critical for DNA damage response and apoptosis. (A) Representative immunohistochemical staining shows increased POLD1 expression in human colon tumors than normal human colon tissues. Image credit: Human Protein Atlas. (B) Immunoblotting blot analysis and (C) MTT assay in RKO cells with or without stable POLD1 knockdown. (D) Immunoblotting blot analysis in colon-derived HCT116, SW480 or RKO cancer cells following different doses of DFO treatment. (E) MTT assay in SW480 and RKO cells treated with 10 □M DFO and/or 100 nM CHK1 inhibitor UCN-01 for 48 hours. (F) MTT assay in SW480 cells treated with 10 μM DFO and/or 100 nM CHK1 inhibitor Prexasertib for 48 hours. (G, H) MTT assay in SW480 and MC38 cells treated with 10 μM DFX, 10 uM ATR inhibitor VE-822 or 100 nM Prexasertib for 48 hours. (I) Immunoblotting blot analysis in MC38 treated 10 uM DFX and/or 100 nM Prexasertib for 48 hours. (J) Immunoblotting blot analysis in SW480 treated 10 uM DFX and/or 10 uM VE-822 for 48 hours. **p<0.01 and ***p<0.01. NS, Not significant.

FIG. S8 shows a working model. (A) Abundant TFRC-mediated iron uptake in colon tumors is required for maintaining the activity of the metal-dependent TNKS, which causes poly-ADP-ribosylation and degradation of Axin2, and activation of β-catenin/c-Myc/E2F1/POLD1 signaling. (B) TFRC deficiency-caused low iron leads to decreased TNKS activity, stabilized Axin2, β-catenin phosphorylation and degradation, suppressed c-Myc/E2F1/POLD1 transcription, increased DNA replicative stress, DNA damage and subsequent cell apoptosis. (C) TFRC is induced by active β-catenin signaling due to genetic APC mutation, whereas TFRC-mediated intratumoral iron accumulation potentiates β-catenin signaling via directly enhancing the activity of TNKS. TFRC-mediated iron import is at the center of this feed-forward loop to facilitate tumor cell survival via supplying nucleotides for DNA damage repair in CRC. Combination of iron chelation and DNA damaging agents had a synergistic effect on inducing DNA damage and suppressing cell growth in CRC.

### DETAILED DESCRIPTION OF THE INVENTION

In accordance with the present invention there may be employed conventional cell culture methods, chemical synthetic methods and other biological and pharmaceutical techniques within the skill of the art. Such techniques are well-known and are otherwise explained fully in the literature.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It is to be noted that as used herein and in the appended claims, the singular forms “a,” “and” and “the” include plural references unless the context clearly dictates otherwise.

The following terms, among others, are used to describe the present invention. It is to be understood that a term which is not specifically defined is to be given a meaning consistent with the use of that term within the context of the present invention as understood by those of ordinary skill.

The term “compound” or “agent”, as used herein, unless otherwise indicated, refers to any specific chemical compound disclosed herein and includes tautomers, regioisomers, geometric isomers as applicable, and also where applicable, optical isomers (e.g. enantiomers) thereof, as well as pharmaceutically acceptable salts thereof (including both acid addition and base addition salts). A pharmaceutically acceptable salt means an acid or base salt of any one or more of the compounds used in the present invention that is of sufficient purity and quality for use in the formulation of a composition or medicament of the present invention and which are tolerated and sufficiently non-toxic to be used in a pharmaceutical preparation, often to increase the aqueous solubility of the compound and enhance its bioavailability. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds as well as diastereomers and epimers, where applicable in context. The term also refers, in context to prodrug forms of compounds which have been modified to facilitate the administration and delivery of compounds to a site of activity.

The term “patient” or “subject” is used throughout the specification within context to describe an animal, generally a mammal and preferably a human, to whom treatment, including prophylactic treatment (prophylaxis), with the methods and compositions according to the present invention is provided. For treatment of those conditions or disease states which are specific for a specific animal such as a human patient, the term patient refers to that specific animal. In the present invention, the patient or subject referred to is often a human cancer patient, although veterinary applications of the present invention and treatment of cancer in domesticated animals is clearly contemplated.

The terms “effective” or “pharmaceutically effective” are used herein, unless otherwise indicated, to describe an amount of a compound or composition which, in context, is used to produce or affect an intended result, whether that result relates to the chelation of iron (for the inhibition of DNA Polymerase) or inhibition of checkpoint kinase 1 (Chk1), the inhibition of ataxia telangiectasia mutated and Rad3 related kinase (ATR) or the promotion of DNA damage or other anticancer effect in the inhibition of cancer, or to potentiate the effects of a concomitant treatment of cancer (synergism). This term subsumes all other effective amount or effective concentration terms (including the term “therapeutically effective”) which are otherwise described in the present application.

The terms “treat”, “treating”, and “treatment”, etc., as used herein, refer to any action providing a benefit to a patient at risk for or afflicted by a cancer. Treatment, as used herein, encompasses both prophylactic (reducing the likelihood of an adverse effect occurring) and therapeutic treatment (inhibiting or even reversing the adverse effect). Favorable or successful treatment of cancer includes inhibiting and/or reversing, ameliorating, reducing the effect of one or more symptoms of cancer. Favorable prophylaxis includes reducing the likelihood and/or preventing in certain patients or subjects the formation of cancer or metastasis of cancer by pre-treating or co-administering an iron chelator and a CHK1 inhibitor, an ATR inhibitor, a DNA damaging agent and/or other anticancer agent as described herein.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated.

As used herein, the term neoplasia is used to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. In certain preferred aspects, the cancer which is treated is colorectal cancer, lung cancer, breast cancer, ovarian cancer and/or prostate cancer. In other embodiments, the cancer is breast, ovarian, prostate, cervical (including during pregnancy), testicular, head and neck cancer, Hodgkin's lymphoma, non-small cell lung cancer, lymphoma, brain cancer, neuroblastoma, leukemia, solid tumors, cancer of the bladder, stomach, thyroid, soft tissue sarcoma, multiple myeloma, colon cancer, esophageal cancer, stomach cancer or pancreatic cancer. Often, in embodiments, the cancer is breast, bladder, cervical, colon, head and neck, Hodgkin lymphoma, liver, lung, renal cell, skin, stomach, rectal cancer or any solid tumor which are able to repair errors in its DNA that occur when the DNA is copied. In other embodiments, the cancer is unable to repair errors in its DNA that occur when its DNA is copied.

As used herein, the terms malignant neoplasia and cancer are used synonymously to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma, non-melanoma skin cancer (especially basal cell carcinoma or squamous cell carcinoma), acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention.

Neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms (cancer) are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, stomach and thyroid; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991). All of these neoplasms may be treated using compounds according to the present invention.

Representative common cancers to be treated with compounds according to the present invention include, for example, prostate cancer, metastatic prostate cancer, stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by compositions according to the present invention. Because of the activity of the present compounds, the present invention has general applicability treating virtually any cancer in any tissue, thus the compounds, compositions and methods of the present invention are generally applicable to the treatment of cancer and in reducing the likelihood of development of cancer and/or the metastasis of an existing cancer.

In certain particular aspects of the present invention, the cancer which is treated is metastatic cancer, a recurrent cancer or a drug resistant cancer, especially including a drug resistant cancer. Separately, metastatic cancer may be found in virtually all tissues of a cancer patient in late stages of the disease, typically metastatic cancer is found in lymph system/nodes (lymphoma), in bones, in lungs, in bladder tissue, in kidney tissue, liver tissue and in virtually any tissue, including brain (brain cancer/tumor). Thus, the present invention is generally applicable and may be used to treat any cancer in any tissue, regardless of etiology.

The term “tumor” is used to describe a malignant or benign growth or tumefacent.

The term “checkpoint kinase I inhibitor” or Chk1 inhibitor is used to describe compounds which inhibit the protein checkpoint kinase I, involved in DNA damage repair. This protein belongs to the Ser/Thr protein kinase family and is required for checkpoint mediated cell cycle arrest in response to DNA damage or the presence of unreplicated DNA. This protein acts to integrate signals from ATM and ATR, two cell cycle proteins involved in DNA damage responses, that also associate with chromatin in meiotic prophase I. Phosphorylation of CDC25A protein phosphatase by this protein is required for cells to delay cell cycle progression in response to double-strand DNA breaks. These inhibitors limit and/or prevent cells from repairing DNA damage. Several alternatively spliced transcript variants have been found for this gene. Exemplary Chk1 inhibitors for use in the present invention include, for example, prexasertib, UCN-01, SRA737, AZD7762, rabusertib (LY2603618), MK-8776 (SCH 900776), CHIR-124, PF-477736, VX-803 (M4344), GDC-0575 (ARRY-575), PD0166285, CCT245737 (SRA737, PNT-737), SAR-020106, SB 218078, prexasertib, UCN-01, SRA737, AZD7762, rabusertib (LY2603618), MK-8776 (SCH 900776), CHIR-124, PF-477736, VX-803 (M4344), GDC-0575 (ARRY-575), PD0166285, SAR-020106, SB 218078, TCS2312, aminothiadiazole and aminothiadiazole conjugated cyanopyridines, CCT245737 (SRA737, PNT-737), CCT245737(S), CCT244747, GDC-0425, GNE-783, GNE-900, GO-6976, MU-380, NSC30049, PD0407824, PD-321852, V158411 or a pharmaceutically acceptable salt or mixture thereof.

The term “ataxia telangiectasia mutated and Rad3 related kinase inhibitor” or “ATR inhibitor” is used to describe a compound which inhibits ataxia telangiectasia mutated and Rad3-related kinase or ATR, which is involved in DNA damage repair. ATR inhibitors inhibit the growth of tumor cells by limiting their ability to repair damaged DNA. ATR inhibitors work similarly to PARP inhibitor. Exemplary ATR inhibitors for use in the present invention include VE-822 (VX-970, M6620), Elimusertib (BAY1895344), VX-803, EPT-46464, AZ20, ceralasertib (AZD 6738) and VE-821, CGK 733, schisandrin B, HAMNO and Torin 2, among others.

The term “additional anti-cancer compound”, “additional anti-cancer drug” or “additional anti-cancer agent” is used to describe any compound (including its derivatives) which may be used to treat cancer through a mechanism which is other than through DNA damage. The “additional anti-cancer compound”, “additional anti-cancer drug” or “additional anti-cancer agent” can be an anticancer agent which is distinguishable from a CHK1 or ATR inhibitor compound or DNA damaging anticancer agent otherwise used as a chemotherapy/cancer therapy agent in compositions and methods described herein. In many instances, the co-administration of another anti-cancer compound according to the present invention results in a synergistic anti-cancer effect. Exemplary anti-cancer compounds for co-administration with formulations according to the present invention include microtubule inhibitors (e.g., taxol), as well as tyrosine kinase inhibitors (e.g., surafenib), EGF kinase inhibitors (e.g., tarceva or erlotinib) and tyrosine kinase inhibitors or ABL kinase inhibitors (e.g. imatinib), among others.

The term “additional anti-cancer compound”, “additional anti-cancer drug” or “additional anti-cancer agent” is used to describe any compound (including its derivatives) which may be used to treat cancer. The “additional anti-cancer compound”, “additional anti-cancer drug” or “additional anti-cancer agent” can be an anticancer agent such as a taxane, vinca alkaloid and/or radiation sensitizing agent otherwise used as chemotherapy/cancer therapy agent. In many instances, the co-administration of another anti-cancer compound according to the present invention results in a synergistic anti-cancer effect. Exemplary anti-cancer compounds for co-administration with formulations according to the present invention include microtubule inhibitors (e.g., taxol), as well as tyrosine kinase inhibitors (e.g., surafenib), EGF kinase inhibitors (e.g., tarceva or erlotinib) and tyrosine kinase inhibitors or ABL kinase inhibitors (e.g. imatinib).

Anti-cancer compounds for co-administration include, for example, agent(s) which may be co-administered with compounds according to the present invention in the treatment of cancer. These agents include chemotherapeutic agents and include one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurn, vandetanib, ARQ-197, MK-0457. MLN8054, PHA-739358, R-763, AT-9263, a FLT-3 inhibitor, a VEGFR inhibitor, an EGFR TK inhibitor, an aurora kinase inhibitor, a PIK-1 modulator, a Bel-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin. ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib, PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolylj-quinolone, vatalanib. AG-013736. AVE-0005, the acetate salt of [D-Ser(But) 6,Azgly 10] (pyro-Glu-His-Trp-Ser-Tyr-D-Ser(But)-Leu-Arg-Pro-Azgly-NH₂ acetate IC₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU 11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxy urea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deoxyuridine, cytosine arabinoside, 6-mecaptopurine, deoxycoformycin, calcitriol, valrubicin, mithramycin, vinblastine, vinorelbine, topotecan, razoxin, marimastat. COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, ipilimumab, nivolomuab, pembrolizumab, dabrafenib, trametinib and vemurafenib among others.

The term “DNA damaging agent” is used to describe an anticancer compound which causes damage to DNA in at least one aspect, and in most instances, as a principle aspect of its mechanism for use in the treatment of cancer. DNA damaging agents include DNA reactive agents, antimetabolites, topoisomerase inhibitors, nitrogen mustard compounds, folate antagonists and alkylating agents, among others. Specific DNA damaging agents which find use in the present invention include the following:
Melphalan
Cyclophosphamide
Temozolomide
Carmustine (BCNU)
Fotemustine
Fotemustine
Ecteinascidin-743
Duocarmycin A
Temozolomide
Dacarbazine
Laromustine
Duocarmycin A
Mithramycin A
Duocarmycin A
CC-1065
Adozelesin
Carzelesin
Bizelesin
Tallimustine
Cisplatin
Fotemustine
Diflometecan
Nitrogen mustards:
Mechlorethamine
Chlorambucil
Bendamustine
Spiromustine
Uramustine
Estramustine phosphate
DHEA mustard
Prechimustine
Imidazole mustard
Cyclophosphamide
Ifosfamide
Trofosfamide
Mafosfamide
Nitrosoureas:
SarCNU
Iomustine (CCNU)
Lomustine
Semustine
Ranimustine
Nimustine
Streptozotocin
Chlorozotocin
1,2-bis(sulfonyl)hydrazine
Procarbazine
p-methylbenzylhydrazine
Thiotepa
Triethylene melamine
Hexamethylmelamine
Pentamethylmelamine
Triaziquone
Carbazilquinone
AZQ (Diaziquinone)
BZQ
DZQ
Busulfan
Dimethylbusulfan
Treosulfan
Hepsulfan
Mannosulfan
Methylene dimethane sulfonate
Illudin A
Illudin B
Illudin M
Illudin S
HMAF
Cisplatin
Carboplatin
Nedaplatin
Iproplatin
Oxaloplatin
Lobaplatin
Strataplatin
Spiroplatin
Picoplatin
Heptaplatin
Triplatin tetranitrate
Ecteinascidin-743
Indicine-N-oxide
Dianhydrogalactitol
Diacetyl dianhydrogalactitol
Teroxirone
Adolzelesin
CC-1065
Carzelesin
Duocarmycin-A
Ducarmycin-SA
Bizelesin
KW 2189
Distamycin
Tallimustin
MEN 10710
MEN 10716
Brostalicin
PNU-151807
Mithramycin A
Mithramycin SK
Chromomycin A
Chromocyclomycin
Olivomycin
UCH9
Mitomycin
Streptonigrin
Porfirmycin
KW-2149
Daunorubin
Doxorubin
Idarubin
Amrubicin
Epirubicin
Valrubicin
Pirarubicin
Berubicin
Carubicin
Esorubicin
Detorubicin
Duborimycin
Zorubicin
Aclacinomycin
Marcellomycin
Musettamycin
Ametantrone
Mitoxantrone
Pixantrone
Teloxantrone
Piroxantrone
Losoxantrone
Bisantrene
Calicheamicin
Esperamicin
Namenamicin
Shishjimicin
Neocarinostatin
Lidamycin
Kedarcidin
Maduropeptin
N1999A2
Actinomycin D
Bleomycin
Fostriecin
Irinotecan
Rubitecan
Exatecan
Kareniticin
Topotecan
Lurtotecan
CKD602
Camptothecin
Gimatecan
Diflomatecan
Rebeccamycin
NB-506
Edotecarin
AT2433
Lamellarin A
Lamellarin C
Lamellarin D
Lamellarin G
Lamellarin L
Lamellarin M
NSC 314622
NSC 706744
NSC 726972
NSC 725766
NSC 724988
NSC 727357
Etoposide
Teniposide
Chloroquinoxoline sulfonamide
XK469
Asulacrine
Razoxane
Elliptinium
Amonafide
Batracylin
Aminopterin
Methotrexate
Pralatrexate
Edatrexate
Talotrexin
Pemetrexed
Ralitrexed
Nolatrexted
OSI-7904L
Thymectacin
Lonatrexole
Dapsone
AG2034
Triazinate
Trifluridine
5-Fluorouracil
5-iododeoxyuridine
5-bromodeoxyuridine
6-azaurdine
Cytarabine
Fazarabine
Decitabine
Gemcytibine
Sapacitabine
Penclomedine
m-azidopyramethamine
metoprine
hydroxyurea
6-mercaptopurine
6-thioguanine
Azathioprine
Methylthioinosine
Thioguanine deoxyriboside
Cladribine
Vidarabine
2′-deoxycoformicin
Fludarabine
Clofarabine
Nelarabine
L-alanosine
Forodesine
Inosine dialdehyde
Tiazofurin
Triapine

The term “radiation therapy” or “radiotherapy” is used to describe an ancillary or alternative anticancer therapy which can be used in combination with iron chelators to produce synergistic anticancer effects in patients. Radiation therapy is a therapy using ionizing radiation to treat cancer, generally provided as part of treatment to control or kill malignant cells and is delivered by one or more methods, as described herein below. Radiation therapy may be curative in a number of types of cancer if they are localized to one area of the body, but in the present invention is used in combination with an iron chlelator as described herein. Radiotherapy may also be used in combination therapy pursuant to the present invention to prevent tumor recurrence after surgery to remove a primary malignant tumor (for example, early stages of breast cancer). Radiation therapy is synergistic in combination with iron chelators pursuant to the present invention. Radiation therapy is often used against cancerous tumors because of its ability to control cell growth, principally by damaging the DNA of cancerous tissue leading to cell death. To spare normal tissue shaped radiation beams may be aimed from several angles of exposure to intersect at the tumor, providing a much larger absorbed dose than in the surrounding healthy tissue. Besides the tumour itself, the radiation may also be used to treat draining lymph nodes if they are clinically or radiologically involved with the tumor, or if there is thought to be a risk of subclinical malignant spread. The precise treatment intent (curative, adjuvant, neoadjuvant therapeutic or palliative) will depend on the tumor type, location, and stage, as well as the general health of the patient. Total body irradiation (TBI) is a radiation therapy technique used to prepare the body to receive a bone marrow transplant. Brachytherapy is another form of radiation therapy in which a radioactive source is placed inside or next to the area requiring treatment. This approach minimizes exposure to healthy tissue during procedures to treat cancers of the breast, prostate and other organs. Often the principal types of radiotherapy used in the present invention includes external beam radiation therapy, contact x-ray brachytherapy, brachytherapy (sealed source radiotherapy), radionuclide therapy and/or intraoperative radiotherapy.

The term “co-administration” or “combination therapy” is used to describe a therapy in which at least two active compounds in effective amounts are used to treat cancer as otherwise described herein, either at the same time or within dosing or administration schedules defined further herein or ascertainable by those of ordinary skill in the art. Although the term co-administration preferably includes the administration of two active compounds to the patient at the same time (contemporaneously, concominantly or sequentially), it is not necessary that the compounds be administered to the patient at exactly same time, although effective amounts of the individual compounds will be present in the patient at the same time. In addition, in certain embodiments, co-administration will refer to the fact that two compounds are administered at significantly different times, but the effects of the two compounds are present at the same time. Thus, the term co-administration includes an administration in which the active agents (e.g. the Chk1 inhibitor, ATR inhibitor, or DNA damaging agent or other anticancer compound in combination with an iron chelator compound) are administered for example, at approximately the same time (contemporaneously) or at different times ranging from about one to several minutes to about eight hours or longer, about 30 minutes to about 6 hours or about an hour to about 4 hours.

Co-administration of the iron chelator compound and a Chk1 inhibitor, ATR inhibitor or a DNA damage agent or other anticancer agent pursuant to the present invention unexpectedly produces a synergistic enhancement of the anticancer activity of the two agents. The term “synergistic” is used pursuant to the present invention to describe an anti-cancer effect which occurs from the administration of an iron chelator compound and a Chk1 inhibitor, ATR inhibitor, a DNA damaging agent or other anticancer, or mixtures thereof, which is greater than an additive effect that one would expect from the administration of the combination of compounds. Often the combination of agents which are administered to a patient with cancer produces a synergistic (more than additive) anticancer effect. The iron chelator compound and Chk1 inhibitor, ATR inhibitor or DNA damaging agent or other anticancer agent may also be co-administered with another bioactive agent (e.g., antiviral agent, antihyperproliferative disease agent, agents which treat chronic inflammatory disease, etc.).

Pharmaceutical compositions comprise combinations of an effective amount of at least one iron chelator compound as disclosed herein, in combination with at least one or more of a Chk1 inhibitor, ATR inhibitor, or a DNA damage agent in effective amounts to provide synergistic anti-cancer activity in compositions according to the present invention. In addition, one or more other additional anti-cancer compounds as otherwise described herein, all in effective amounts, may be included in pharmaceutical compositions according to the present invention. Each composition may further (preferably) include a pharmaceutically effective amount of a carrier, additive and/or excipient.

The compositions used in methods of treatment of the present invention, and pharmaceutical compositions of the invention, may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat, among others.

The compositions used in methods of treatment of the present invention, and pharmaceutical compositions of the invention, may be administered orally, parenterally, by inhalation spray, topically, rectally, nasally, buccally, vaginally or via an implanted reservoir, among others. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions used in methods of treatment of the present invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially to treat skin cancers. Suitable topical formulations are readily prepared for each of these areas or organs. Topical application for the lower intestinal tract can be effected in a rectal suppository formulation (see above) or in a suitable enema formulation.

Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition of the instant invention that may be combined with the carrier materials to produce a single dosage form will vary depending upon the host and the type of cancer treated, and the particular mode of administration. Preferably, the compositions should be formulated to contain between about 0.05 milligram to about 750 milligrams-1 gram or more, more preferably about 1 milligram to about 600 milligrams, and even more preferably about 10 milligrams to about 500 milligrams of at least one iron chelator compound and at least one or more of a Chk1 inhibitor, ATR inhibitor, DNA damaging agent further optionally in combination with at least one additional anti-cancer active ingredient. In alternative embodiments, the pharmaceutical composition comprises at least one iron chelator compound in combination with at least one Chk1 inhibitor compound, ATR inhibitor, DNA damaging agent, further optionally in combination with at least one additional anti-cancer active agent all in effective amounts to reduce the likelihood, inhibit or reverse cancer in a patient or subject in need.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

These and other aspects of the invention are described further in the following non-limiting examples, which are presented herein.

### EXAMPLES

### Experimental Overview

Transferrin receptor (TFRC) is the major mediator for iron entry into a cell. Under excessive iron conditions, TFRC is expected to be downregulated via the iron regulatory protein and the iron-responsive element machinery to reduce iron uptake and toxicity. However, the mechanism by which the expression of TFRC is maintained high in iron-enriched cancer cells and its contribution to cancer development are enigmatic. In the following experiments, the regulation and function of TFRC-mediated iron uptake in colon were tested in mouse models with colon-specific TFRC disruption and colon-derived cell lines. Transcriptome analysis of patient-derived tumor colonoids was performed to identify molecular targets of iron.

The experiments described herein below shows that TFRC is induced by adenomatous polyposis coli gene loss-driven β-catenin activation in colorectal cancer, whereas TFRC-mediated intratumoral iron accumulation potentiates β-catenin signaling via directly enhancing the activity of tankyrase. TFRC-mediated iron import is at the center of this novel feed-forward loop to facilitate colonic epithelial cell survival. Mechanistically, disruption of TFRC led to a reduction of colonic iron levels and iron-dependent tankyrase activity, which caused stabilization of Axin2 and subsequent repression of the β-catenin/c-Myc/E2F1/DNA polymerase delta1 (POLD1) axis. POLD1 knockdown, iron chelation and TFRC disruption increased DNA replication stress, DNA damage response, apoptosis and reduced colon tumor growth. Strikingly, a combination of iron chelators and DNA damaging agents caused a synergistic effect in inducing DNA damage response and reducing colon tumor cell growth.

As shown by the experiments described herein, together, the TFRC/iron/tankyrase/Axin2/β-catenin/c-Myc/E2F1/POLD1 axis is essential for colon homeostasis and provide novel strategies for colorectal cancer therapy. Here it is found that TFRC is required for maintaining colon homeostasis. High expression of TFRC increased iron uptake and accumulation in CRC, whereas TFRC disruption caused iron reduction in colons. Transcriptomics analysis identified that iron chelation reduced iron-sulfur protein DNA polymerase delta 1 (POLD1). Mechanistic study revealed that iron was required for the activity of tankyrase (TNKS), which can cause degradation of Axin2 and activate β-catenin signaling. In contrast, iron chelation caused downregulation of β-catenin target gene c-Myc, which subsequently reduced the transcription factor E2F1 and its target gene POLD1. Similar to iron chelation and TFRC disruption, POLD1 reduction caused increased DNA replication stress, DNA damage response (DDR), apoptosis and repressed tumor growth. Strikingly, a synergistic effect was found between iron restriction and inhibition of DNA damage signaling proteins, which provides a potential desirable strategy for CRC treatment.

### Materials and Methods

### Cell Culture

Human HCT116, RKO, SW480, HEK293T and murine MC38 CRC cells were maintained at 37° C. in 5% CO₂ and cultured in Dulbecco's modified Eagle medium (DMEM) supplemented with 10% fetal bovine serum (FBS) and 1% penicillin and streptomycin (VWR, Radnor, PA). HCT116 p53+/+ and p53−/− cells were a kind gift from Professor Bert Vogelstein at the Johns Hopkins University. For generating stable cell lines, MC38 cells were transfected with a mouse shTFRC plasmid (TRCN0000375695, Sigma, St. Louis, MO) or a mouse shPOLD1 plasmid (TRCN0000428858, Sigma), RKO cells were transfected with a human shPOLD1 plasmid (TRCN0000007924, Sigma) and selected with 1 μg/ml puromycin.

### Human CRC Tissues

For the human CRCs and adjacent normal colon tissues, RNAs, proteins, frozen sections were prepared from banked snap frozen surgical resection tissues present in the UNM Cancer Center Human Tissue Repository & Tissue Analysis Shared Resource. The UNM Institutional Review Board approved this study (#19-131).

### Animals

All mice were maintained in a standard cage in light and temperature-controlled room and were allowed standard chow and water except as indicated. Animal studies were performed in accordance of the Institute of Laboratory Animal Resources guidelines and approved by the Institutional Animal Care and Use Committee (IACUC) at the University of New Mexico Health Sciences Center (Protocol #HSC-18-200699, 20-201060-HSC) and followed the National Institutes of Health guide for the care and use of Laboratory animals (NIH Publications No. 8023, revised 1978). Tfrc floxed (Tfrc^{F/F}) mice (Stock No: 028177) and mice with the tamoxifen (TAM)-inducible caudal type homeobox 2 (CDX2)^{ERT2}-Cre promoter (Stock No: 022390) were both purchased from the Jackson lab and crossed to generate colon-specific TFRC knockout (CDX2^{ERT2} Tfrc^{F/F}) mice. For fluorescein isothiocyanate (FITC)-dextran gut permeability assay, CDX2^{EFT2} Tfrc^{F/F} and Tfrc^{F/F} mice were treated with 100 mg/kg TAM for 3 days and 7 days later were given by oral gavage with 600 mg/kg FITC-dextran. For colitis study, CDX2^{ERT2} Tfrc^{F/F} and Tfrc^{F/F} mice were treated with 100 mg/kg TAM for 3 days and 7 days later were given with regular chow and 3% dextran sodium sulfate (DSS) water or a low iron diet (3.5 mg/kg iron, 3.5Fe, Research Diets, Inc., New Brunswick, NJ) and 2% DSS water for 7 days. For the survival analysis, CDX2^{ERT2}Apc^{F/F} and CDX2^{ERT2} Tfrc^{F/F} Apc^{F/F} mice were generated and treated with 100 mg/kg TAM for 3 consecutive days and 7 days later were treated with 1.5% DSS for 7 days and then were put back to regular drinking water. For the qPCR and immunoblotting analysis, CDX2^{ERT2} Apc^{F/F} and CDX2^{ERT2} Tfrc^{F/F} Apc^{F/F} mice were treated with 100 mg/kg TAM for 3 consecutive days and sacked 7 days later. For establishing CRC model, CDX2^{T2}Apc^{F/+} and CDX2^{ERT2} Tfrc^{F/F} Apc^{F/+} were generated and treated with 2% DSS for 7 days (inflammatory phase) and then were put on regular drinking water for 14 days (recovery phase). One more inflammatory phase and recovery phase were performed. For subcutaneous xenograft study, 1×10⁶ syngeneic MC38 cells with Tfrc or Pold1 knockdown were injected into the flanks of C57BL/6 mice. Two weeks later, mice were sacrificed and tumors were collected. For combination therapy, when tumors are palpable at one week after subcutaneous injection of MC38 cells, C57BL/6 mice were treated with vehicle, 20 mg/kg DFX for every other day, 1 mg/kg 5-fluorouracil (5-FU) daily, or the combination of DFX and 5-FU.

### Immunoblotting Analysis

Cells and tumor tissues were lysed with radioimmunoprecipitation assay buffer. After incubation, cell extracts were centrifuged and the supernatant was collected for Bradford assay to quantify protein concentration by a BioTek Synergy HTX Multi-Mode Microplate Reader (BioTek, Winooski, VT). Equal amounts (20-50 μg) of protein were loaded for sodium dodecyl sulfate polyacrylamide gel electrophoresis (SDS-PAGE). The proteins inside the gels were transferred onto nitrocellulose membrane with a wet transfer method. The membranes were blocked with 3% milk for 1 hour. Primary antibodies were incubated overnight. Secondary antibodies were incubated for 1 hour. Antibodies for ferritin heavy chain (FTH1, #3998), IRP2 (#37135), active beta catenin (#8814), Anti-rabbit IgG HRP-linked Antibody (#7074) and Anti-mouse IgG HRP-linked Antibody (#7076) were from Cell signaling Technology (Danvers, MA). Primary antibodies for TFRC (sc-393719), HIF-1α, (sc-13515), HIF-2a (sc-13596), pS6 (sc-514033), pS6K (sc-8418), RRM2 (sc-398294), p-p53 (sc-377567), p53 (sc-6243), c-MYC (sc-40), GAPDH (sc-47724), Cyclin D1 (sc-718) and Actin (sc-8432) were from Santa Cruz Biotechnology (Dallas, TX). p-RPA2 (PA5-39809) was from Invitrogen (Carlsbad, CA). POLD1 (15646-1-AP), p-CHK1 (28805-1-AP), E2F1 (66515-1-Ig), Axin2 (20540-1-AP) and TNKS (18030-1-AP) were from Proteintech (Rosemont, IL).

### Patient-Derived Colorectal Tumor Colonoids

The adenoma colonoids culture was described previously. See Xue, et al., Cell Metab, 2016; 24: pp. 447-461). The culture plates were placed in a 37° C. incubator for 30 min to solidify the Matrigel, followed by the addition of 1 mL serum-free Keratinocyte Growth Media Gold (KGMG, Catalogue: 00195769, Lonza, Basel, Switzerland). After 24 hours of plating, colonoids were treated with DFO (0 or 100 μM) in KGMG for 4 days with fresh media change every other day. At the end of the experiment, culture media was aspirated off, and colonoids were lysed with Trizol to extract RNA.

### Statistical Analysis

Data were expressed as mean±SD. p values were calculated by independent t-test, paired t-test, one-way and two-way analysis of variance (ANOVA). p<0.05 was considered significant.

### Detailed Methods

### Histology Analysis, Immunofluorescence (IF) and 3,3′-Diaminobenzidine (DAB) Enhanced Ped1's Iron Staining

Paraffin sections were deparaffinized and rehydrated to distilled water. For hematoxylin and eosin (H & E) staining, the sections were incubated with hematoxylin solution 2 minutes and then were washed with tap water for 5 minutes. After washing, the slides were submerged in bluing solution with 1-2 dips. The slides were rinsed with tap water 2 minutes and incubated with Eosin solution for 5 minutes. After incubation, the sections were dehydrated with 95%, 100% Ethanol and xylene, and covered for pathologic examination by a pathologist. For IF staining, the sections were put into 10 mM sodium citrate buffer in a sub-boiling temperature for 12 minutes. The slides were left on bench top for 2h to cool down and then were blocked with 10% normal goat serum (NGS) for 1 h. Primary antibodies were incubated overnight at 4 degrees and secondary antibodies were incubated 1h at room temperature. The sections were mounted with EverBrite™ mounting medium (Biotium, Fremont, CA). For DAB enhanced Perl's iron staining, the sections were incubated in a mixture of 2% hydrochloric acid and 1% ferrocyanide solution (1:1) for 30 minutes. After incubation, the sections were rinsed in tap water for 5 minutes. Then the slides were immersed in 0.05% DAB solution for 20 minutes. The slides were washed with tap water for 5 minutes and then sealed with coverslip using Permount mounting medium (Fisher Scientific, Hampton, NH). Primary antibodies for TFRC (#13113), cleaved Caspase3 (CC3, #9664) and Ki67 (#12202) were from Cell Signaling Technology (Danvers, MA), whereas γH2AX (sc-517348) was from Santa Cruz Biotechnology (Dallas, TX).

### Ferrous Iron Beads Pull Down Assay

The method used for precipitating proteins by ferrous iron beads was described previously⁸. Briefly, metal-free beads or ones with ferrous iron immobilized onto a pentadentate chelator coupled to a quartz base matrix (PDCSLQ free and Fe-PDC-SLQ) were obtained from Affiland (Liege, Belgium). The beads were washed three times (twice the bead bed volume) with EDTA-free Triton lysis buffer (25 mM HEPES, 100 mM NaCl, 10% glycerol, and 1% Triton X-100) to remove excess unbound ferrous iron. The beads were then used to precipitate proteins from HCT116. Thirty microliters of beads were added to 1 mg whole-cell lysates in the Triton lysis buffer and rotated at 4° C. for 2 hr. The beads were then washed three times with Triton lysis buffer, pelleted, and the precipitate was resuspended in 5× loading buffer for immunoblotting.

### FerroOrange Staining

Cells (2×10⁵ cells/well) were plated into 24 well plates. After seeding, pre-warmed DMEM containing 0.5 μM ferroOrange (dojindo, Rockville, MD), was incubated at 37° C. for 30 min. After incubation, the cell images were taken. Representative images were taken using the RFP channel of an Invitrogen™ EVOS™ FL Auto Imaging System (Thermo Fisher Scientific, Waltham, MA). Fluorescence intensity was quantified using a SpectraMax M2 Microplate Reader (Molecular Devices, Radnor, PA) at excitation 543 nm and emission 580 nm. The intensity was normalized with protein concentrations.

### Transient Transfection and Luciferase Assay.

For luciferase assay, cells were seeded into a 24-well plate at a cell density of 5×10⁴ cells per well. TOPflash luciferase constructs were co-transfected with pcDNA3 beta-Catenin S33Y (19286, Addgene), TNKS (Homo sapiens) in pLenti6.3N5-DEST (HsCD00946323, DNASU) by polyethylenimine (PEI; Polysciences Inc., Warrington, PA). HA-E2F-1 wt-pRcCMV (#21667, Addgene), POLD1_pLX307 (#98358, Addgene), pCDH-puro-cMyc (#46970, Addgene) or empty plasmids were transfected into cells using lipofectamine 2000 (Thermo Fisher Scientific). Experiments were carried out 24˜48 hours post-transfection. Similarly, RKO were transfected with TFRC siRNA (M-003941-02-0005) from GE Dharmacon (Lafayette, CO), and SW480 were transfected with c-Myc siRNA (EHU021051), E2F1 siRNA (EHU070981), TNKS siRNA (EHU142711) and AXIN2 siRNA (EHU001481) from Millipore (Burlington, MA).

Quantitative Polymerase Chain Reaction (qPCR) Analysis

Total RNA was extracted using IBI Isolate DNA/RNA Reagent Kit (IB47602, IBI Scientific, Dubuque, IA). qPCR was performed using a LightCycler 480 instrument (Roche Diagnostics, Indianapolis, IN). For DSS treated tissue, RNA was further precipitated with 8M LiCl (⅓ volume of RNA solution) and purified. The used pre-designed primers were listed in Table S1 (see below).

### Rna-Seq and Data Analysis.

RNA sequencing libraries were prepared using the TruSeq RNA library prep kit v2 (Illumina) following the manufacturer's recommended protocol. The libraries were sequenced using single-end 50-cycle reads on a HiSeq 4000 sequencer (Illumina) at the University of Michigan DNA Sequencing Core Facility. Raw sequencing read quality was assessed utilizing FastQC. Reads were aligned to the reference human transcriptome (UCSC) using STAR 2.5.2a. Default parameters were used for the alignment, with the exception of “--outFilterMultimapNmax 10” and “--sjdbScore 2”. Expression quantification and differential expression analysis between DFO and control tumor colonoids were conducted using CuffDiff v 2.1.1 using default settings. For the CuffDiff analysis, we used Genome Reference Consortium Human Build 37 (GRCh37) as the reference genome. Genes were considered differentially expressed between conditions at a false-discovery rate-adjusted p value of less than 0.05. Differentially expressed pathways were identified utilizing The Database for Annotation, Visualization and Integrated Discovery (DAVID, at url: david.ncifcrfgov/home.jsp). KEGG biological pathways and gene ontology biological processes were considered differentially expressed at a p value of less than 0.05.

### Thiazolyl Blue Tetrazolium Bromide (MTT) Assay

Cells were plated at a concentration of 5×10⁴ cells/mL in a 24 well plates. 125 μL 5 mg/mL MTT (Sigma, MO) was added to each plate and incubated for 30 min. Dimethyl sulfoxide (DMSO) was added and absorbance was measured at 570 nm using a BioTek Synergy HTX Multi-Mode Microplate Reader.

### Tissue Iron Assay

Xenograft tumor tissue samples were homogenized in Millipore water (100 μL/10 mg tissue). Lysates were mixed with equal volume of acid solution (1M hydrochloric acid, 10% trichloroacetic acid, 10 g/L ascorbic acid). Then the mixture was incubated 1 hour at 95 degrees. After centrifuge, supernatants were collected and mixed with ferrozine solution (0.5 mM ferrozine, 1.5M sodium acetate and 0.01% mercaptoacetic acid). Absorbance was measured at 562 nm to determine tissue iron levels.

### Crystal Violet Staining

Cells (1×10³ cells per well) were seeded in 6 well plates. After 1 week, the cells were washed with PBS once and fixed in 10% formaldehyde for 10 minutes. Then the cells were stained with 0.05% crystal violet solution for 30 minutes. After incubation, the cells were washed with tap water twice and dried for several minutes. Methanol was added to the plate to solubilize crystal violet in the stained cells. Absorbance was measured at 540 nm.

Supplementary Table 1 qPCR primers.

| | |
|---|---|
| 18S_forward | GTAACCCGTTGAACCCCATT |
| | SEQ ID NO: 1 |
| 18S_reverse | CCATCCAATCGGTAGTAGCG |
| | SEQ ID NO: 2 |
| hTFRC forward: | ACCATTGTCATATACCCGGTTCA |
| | SEQ ID NO: 3 |
| hTFRC reverse: | CAATAGCCCAAGTAGCCAATCAT |
| | SEQ ID NO: 4 |
| mTFRC forward: | TCA AGC CAG ATC AGC AATT CTC |
| | SEQ ID NO: 5 |
| mTFRC reverse: | AGC CAG TTT CAT CTC CAC ATG |
| | SEQ ID NO: 6 |
| hPOLD1 forward: | ATCCAGAACTTCGACCTTCCG |
| | SEQ ID NO: 7 |
| hPOLD1 reverse: | ACGGCATTGAGCGTGTAGG |
| | SEQ ID NO: 8 |
| hRRM2 forward: | GTGGAGCGATTTAGCCAAGAA |
| | SEQ ID NO: 9 |
| hRRM2 reverse: | CACAAGGCATCGTTTCAATGG |
| | SEQ ID NO: 10 |

### Results

### Tfrc is Increased in CRC and is Potentiated by High Iron Diet.

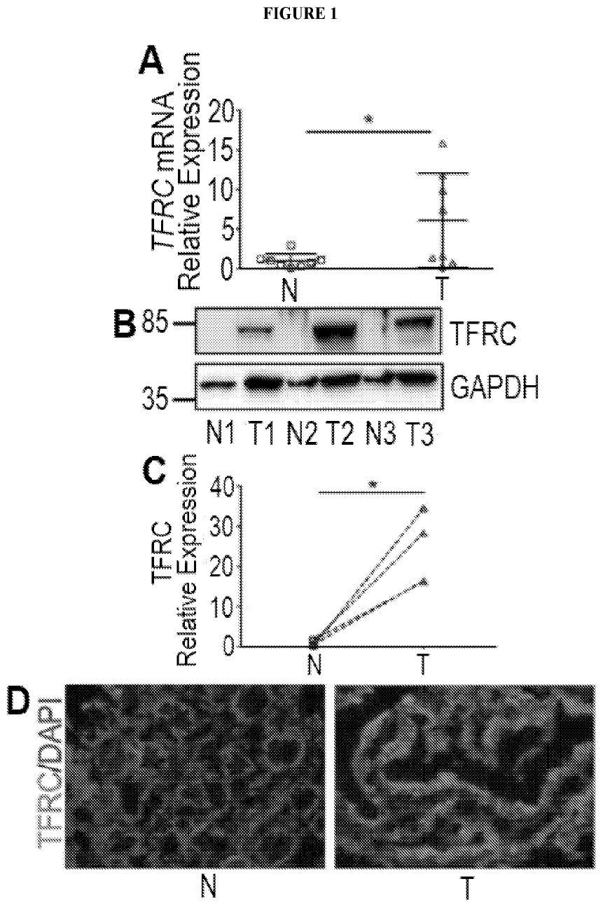

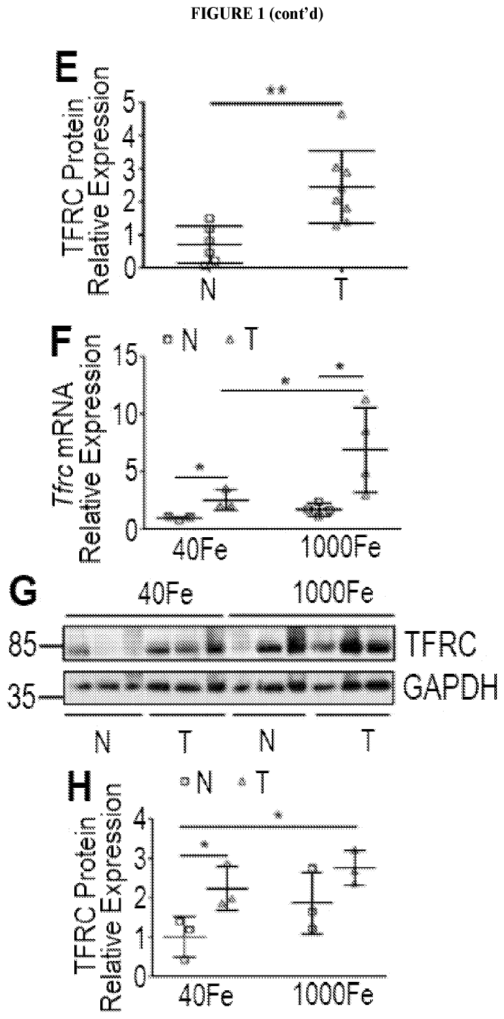

By analyzing the Human Protein Atlas (HPA) database¹⁶, TFRC was mainly expressed in normal epithelial cells (FIG. S1A). In colon tumors, TFRC was expressed both in the cytoplasm and basolateral membrane of epithelial cells (FIG. S1B). Based on UALCAN analysis¹⁷, the mRNA and protein expressions of TFRC from The Cancer Genome Atlas (TCGA) and the Clinical Proteomic Tumor Analysis Consortium (CPTAC) datasets are increased in all stages of colon tumors as compared to normal colons (FIG. S1C-S1F), indicating that TFRC upregulation is an early event during colon tumorigenesis. By qPCR (FIG. 1A), immunoblotting (FIG. 1B, 1C), and immunofluorescence (IF) staining (FIG. 1D, 1E), we confirmed that TFRC was relatively low in normal colons but greatly increased in colon tumors isolated at UNM.

TFRC is modulated by cellular iron level via iron regulatory protein (IRP) and the iron-responsive element machinery¹⁰. To further understand the effect of iron on TFRC in colon tumors, we utilized tissues from colon tumor prone mice that were treated with 1.5% DSS and a high iron diet (1000 mg/kg iron, 1000Fe) or an iron-replete diet (40 mg/kg iron, 40Fe)⁸. Consistently, TFRC was significantly increased in colon tumors than normal colons from 40Fe treated mice (FIG. 1F-1H). Surprisingly, the induction of TFRC in colon tumors was further potentiated by 1000Fe (FIG. 1F-1H). Together, our data showed that TFRC is increased in CRC tissues and high iron diet unexpectedly further enhances this increase in colon tumors.

### Tfrc Disruption Leads to Colon Injury and Increased Susceptibility to Colitis Under Iron Starvation.

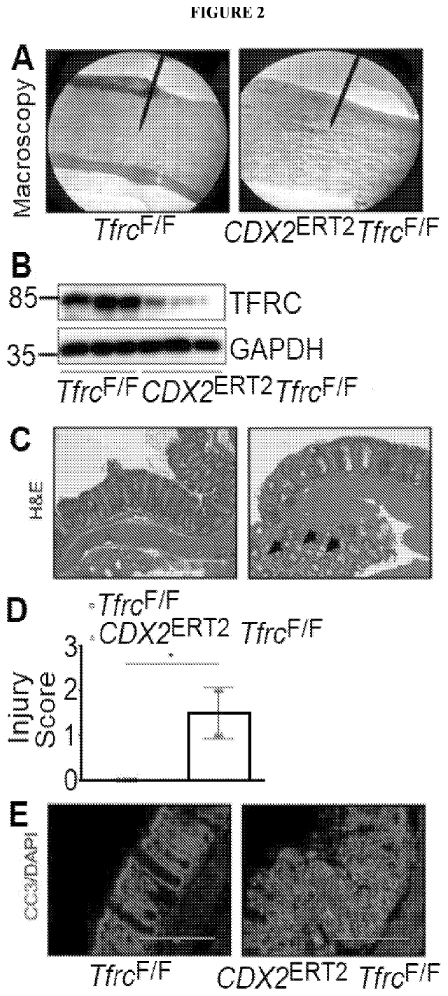

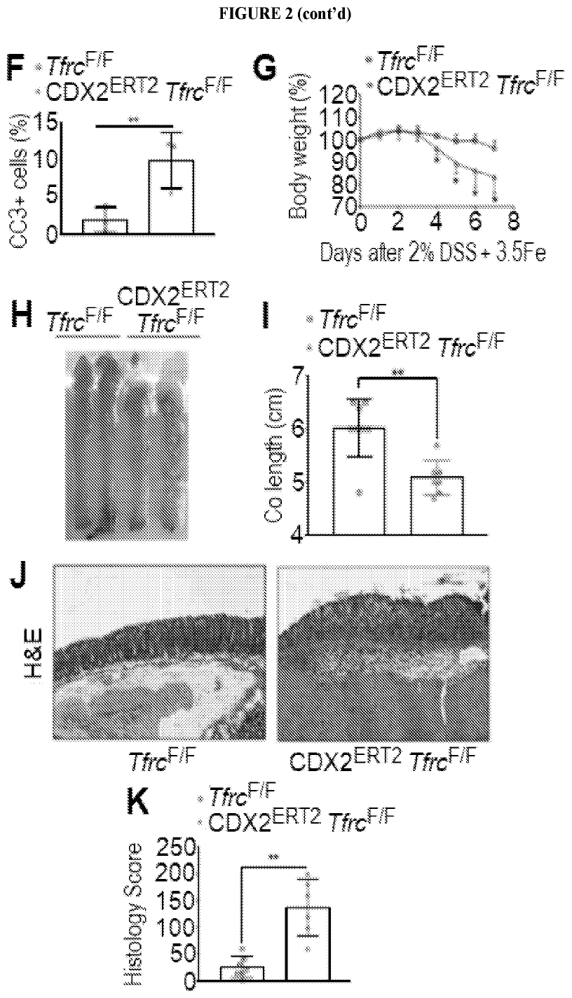

To investigate the role of TFRC in colon, CDX2^{ERT2} Tfrc^{F/F} mice were generated to specifically disrupt TFRC in the colon. The body weights and colon lengths were not changed in CDX2^{ERT2} Tfrc^{F/F} mice after TAM treatment compared to Tfrc^{F/F} mice (FIGS. S2A, S2B). However, TFRC disruption caused a “cobblestone appearance” in the colon under a dissection microscope, indicating tissue damage exists (FIG. 2A). Immunoblotting analysis confirmed the reduction of TFRC in CDX2^{ERT2} Tfrc^{F/F} mice (FIG. 2B). Hematoxylin and eosin (H & E) staining showed that the colon injury was increased in CDX2^{ERT2} Tfrc^{F/F} mice compared to Tfrc^{F/F} mice (FIGS. 2C, 2D). IF staining showed that the cell proliferation marker Ki67 was not changed (FIGS. S2C, S2D), but the apoptosis marker cleaved caspase 3 (CC3) was significantly increased in CDX2^{ERT2} TfrcF/F mice (FIGS. 2E, 2F). However, FITC-dextran assay indicates gut permeability was not changed (FIG. S2E). To examine the impact of the colon injury resulting from TFRC disruption under inflammatory conditions, we treated CDX2^{ERT2} Tfrc^{F/F} mice and Tfrc^{F/F} mice with DSS to induce colitis. We found that the body weights and colon lengths were not different between these two groups (FIGS. S2F, S2G). However, with the co-treatment of DSS and a low iron diet, the body weights were significantly lower and colon lengths were shorter in CDX2^{ERT2} Tfrc^{F/F} mice compared to Tfrc^{F/F} mice (FIG. 2G-21). H&E staining showed that the colonic inflammation was increased in CDX2^{ERT2} Tfrc^{F/F} mice compared to Tfrc^{F/F} mice (FIG. 2J, 2K). Together, these data indicate that TFRC-mediated iron uptake is imperative to cell survival and colon homeostasis especially under iron starvation.

### Tfrc Disruption Prolongs Survival in a Mouse Model of Colon Dysplasia.

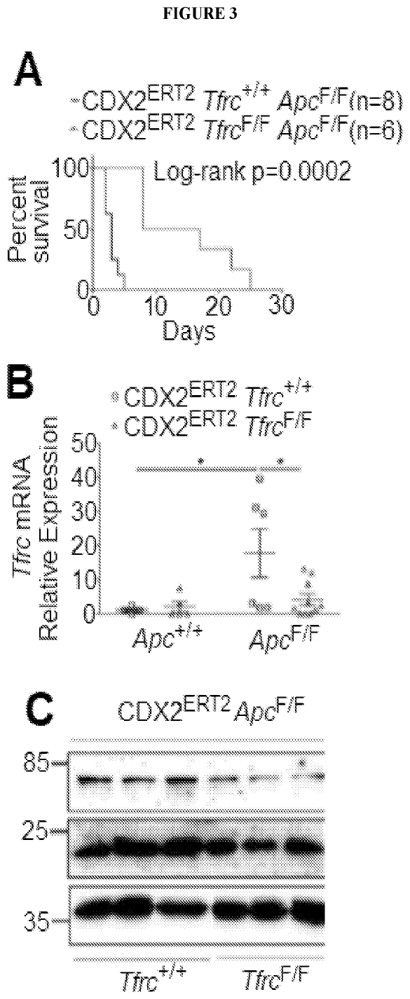

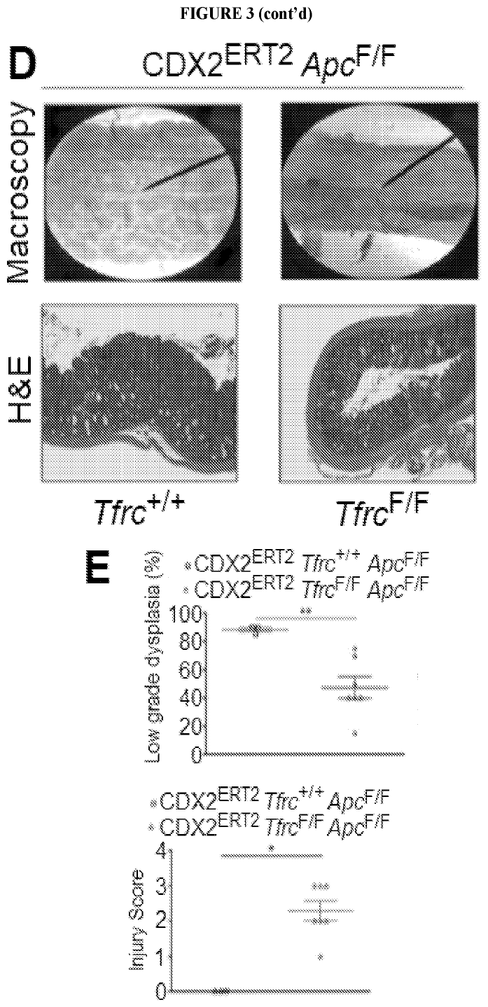

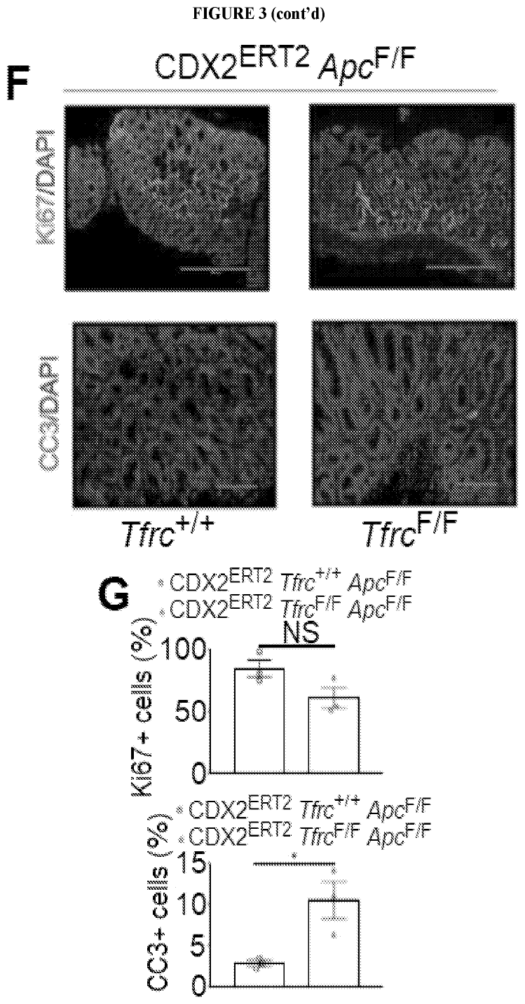

APC is a gene that is mutated in more than 80% of CRC patients¹⁸. To investigate the role of TFRC in colon tumorigenesis, we generated CDX2^{EFT2} Tfrc^{F/F} Apc^{F/F} mice and CDX2^{ERT2} Tfrc^{+/+} Apc^{F/F} mice. After TAM injection and DSS treatment, all the CDX2^{ERT2} Tfrc^{+/+} Apc^{F/F} mice did not live longer than 5 days, while all CDX2^{ERT2} Tfrc^{F/F}ApC^{F/F} mice did (FIG. 3A). To exclude the effects of inflammation, we treated another batch of mice with only TAM. qPCR analysis confirmed that TFRC was significantly induced by Apc depletion, which was blocked by TFRC disruption (FIG. 3B). Immunoblotting analysis demonstrated that TFRC and the cellular iron storage protein FTH1 was reduced in the colons from CDX2^{ERT2} Tfrc^{F/F}Apc^{F/F} mice compared to CDX2^{ERT2} Tfrc^{+/+}Apc^{F/F} mice (FIG. 3C). Macroscopic and histological analysis indicated that TFRC disruption consistently caused more colon injuries but less low-grade dysplasia (FIG. 3D, 3E). IF staining showed that TFRC depletion increased apoptosis but had no effect on cell proliferation (FIG. 3F, 3G). We hypothesized that this TFRC depletion-induced apoptosis was due to reduced iron. Indeed, Perl's iron staining demonstrated that the colonic iron deposit was decreased in CDX2^{ERT2} Tfrc^{F/F} Apc^{F/F} mice (FIG. S3A). Iron deficiency increases cell apoptosis by suppressing mTORC1 signaling¹⁰. Indeed, we found that mTORC1 signaling was significantly reduced in colons from CDX2^{ERT2} Tfrc^{F/F}Apc^{F/F} mice (FIG. S3B). mTORC1 activation is essential for Apc deletion-mediated colon tumorigenesis^{°}. However, the mTORC1 inhibitor rapamycin did not significantly improve survival rate in CDX2^{ERT2} Apc^{F/F} mice (FIG. S3C). Together, these data show that TFRC knockout causes reduced iron accumulation, increased apoptosis, less dysplasia and prolonged survival in this biallelic Apc loss-driven dysplasia in an mTORC1-independent manner.

### Tfrc Depletion Reduces Colon Tumorigenesis by Decreasing Intratumoral Iron.

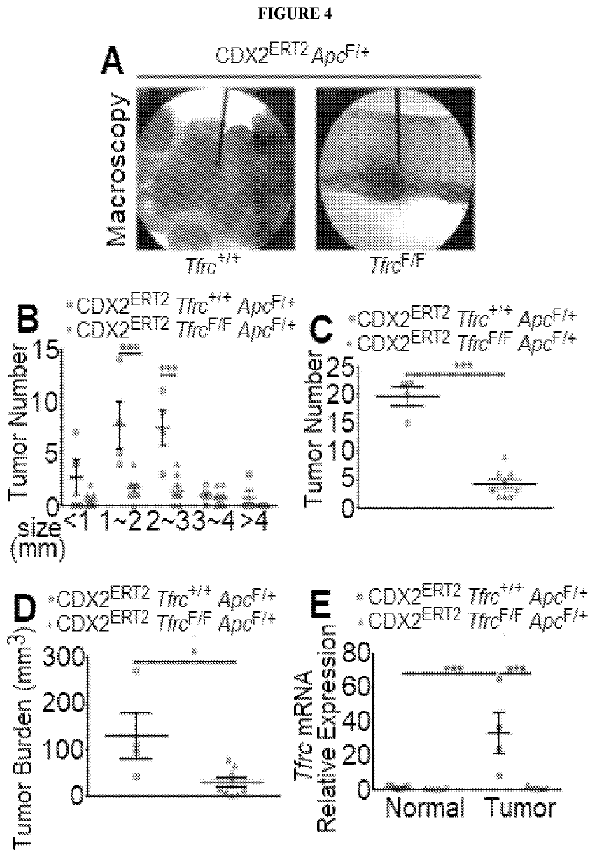

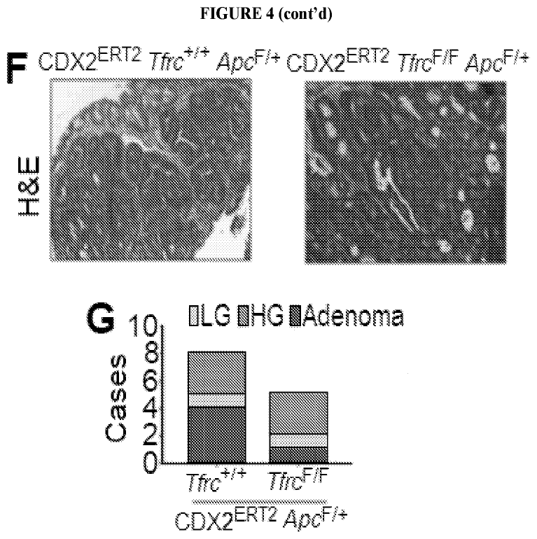

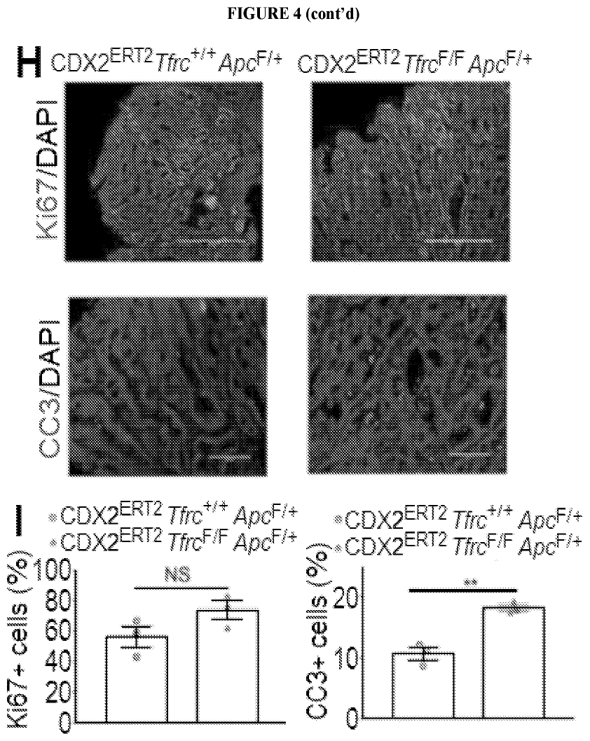

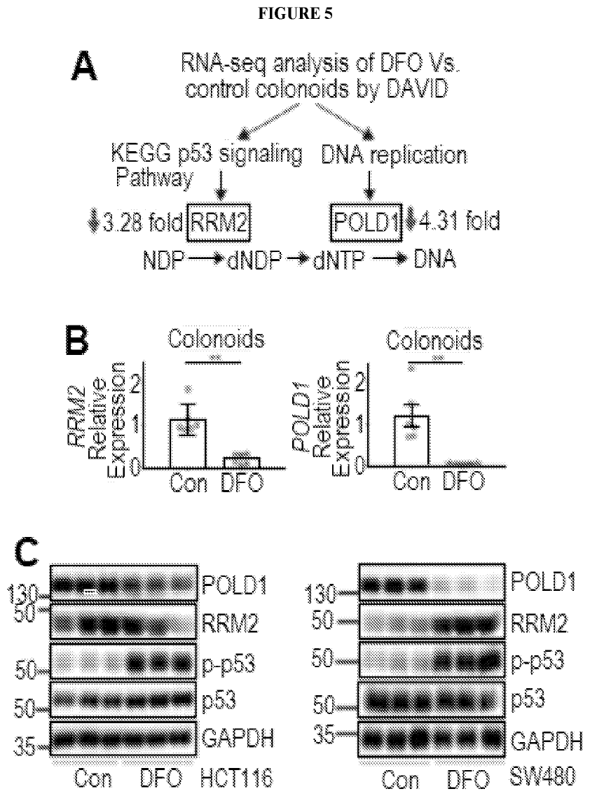

As described above, biallelic Apc deletion leads to colon dysplasia, we further generated mice with monoallelic Apc deletion: CDX2^{ERT2} Tfrc^{F/F} Apc^{F/+} and CDX2^{ERT2} Tfrc^{+/+} Apc^{F/+} mice. After TAM and 2 cycles of DSS treatment, macroscopically visible colon tumors were developed (FIG. 4A). The tumor number at sizes of 1-2 mm and 2-3 mm, total tumor number, and tumor burden were significantly decreased by TFRC depletion (FIG. 4B-4D). qPCR analysis demonstrated that TFRC was significantly increased in the colon tumors compared to normal colons from CDX2^{ERT2} Tfrc^{+/+} Apc^{F/+} mice, whereas TFRC disruption greatly reduced the increased TFRC in colon tumors from CDX2^{ERT2} Tfrc^{F/F} Apc^{F/+} mice (FIG. 4E). Histological analysis found 4 adenomas out of 8 cases in tumors from CDX2^{ERT2} Tfrc⁺⁺ Apc^{F/+} mice, whereas only 1 adenoma out of 5 cases in tumors from CDX2^{ERT2} Tfrc^{F/F} Apc^{F/+} mice (FIG. 4F, 4G). Similarly, IF staining showed that TFRC depletion increased apoptosis but not cell proliferation (FIG. 4H, 4I). TFRC, FTH1 and iron deposition were reduced in colon tumors from CDX2^{ERT2} Tfrc^{F/F}Apc^{F/+} mice (FIGS. S4A, S4B). To further investigate whether TFRC is critical for high iron-driven colon tumorigenesis, we treated mice with 1000 Fe or 40 Fe. Consistently, total tumor number and tumor burden were significantly decreased by TFRC depletion under both diets (FIGS. S4C-S4E). The average tumor sizes were similar across all groups (FIG. S4F). Together, these results indicate that TFRC depletion may reduce colon tumorigenesis by decreasing intratumoral iron.

### The DNA Polymerase POLD1 is Regulated by Iron/TNKS/Axin2/β-Catenin/c-Myc/E2F1 Axis.

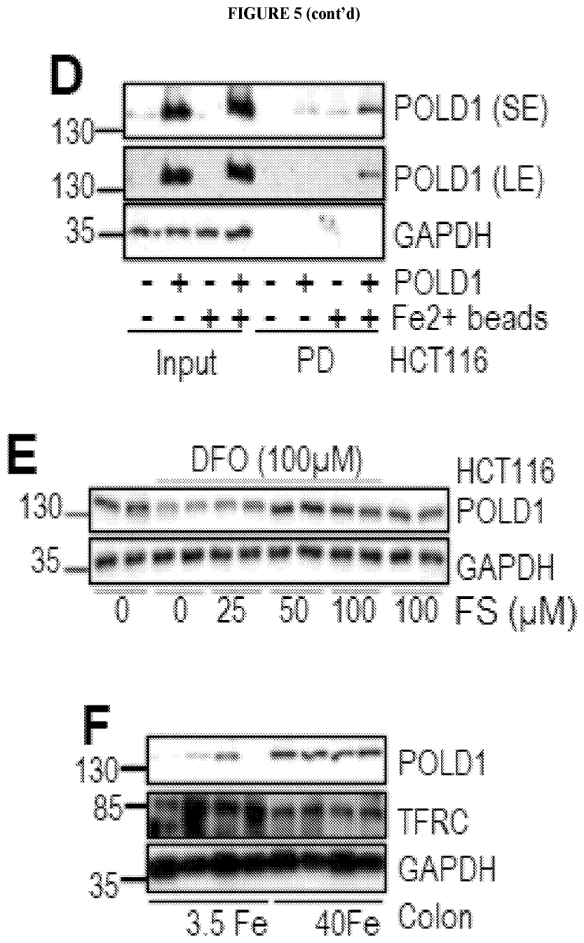

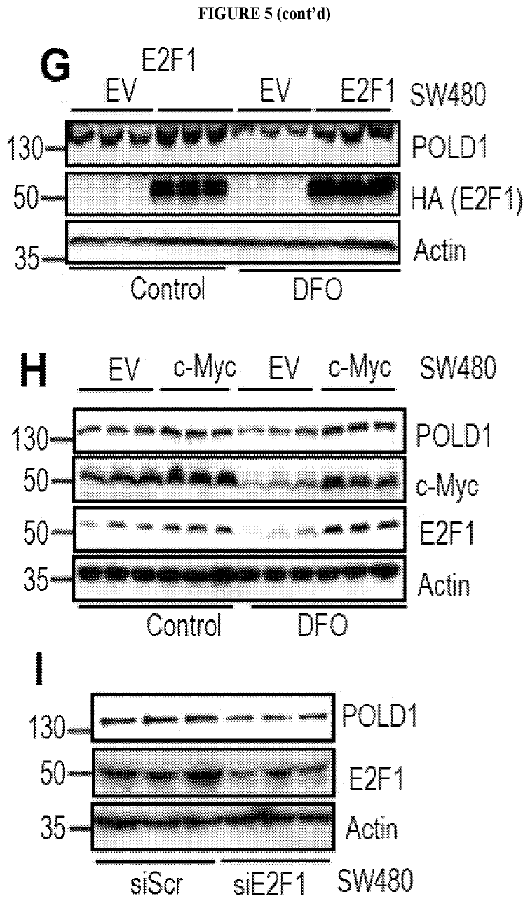

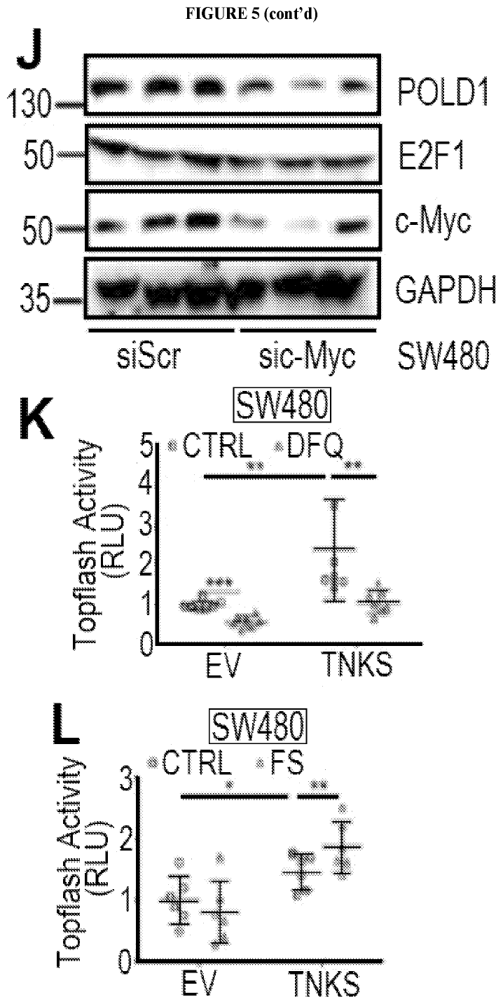

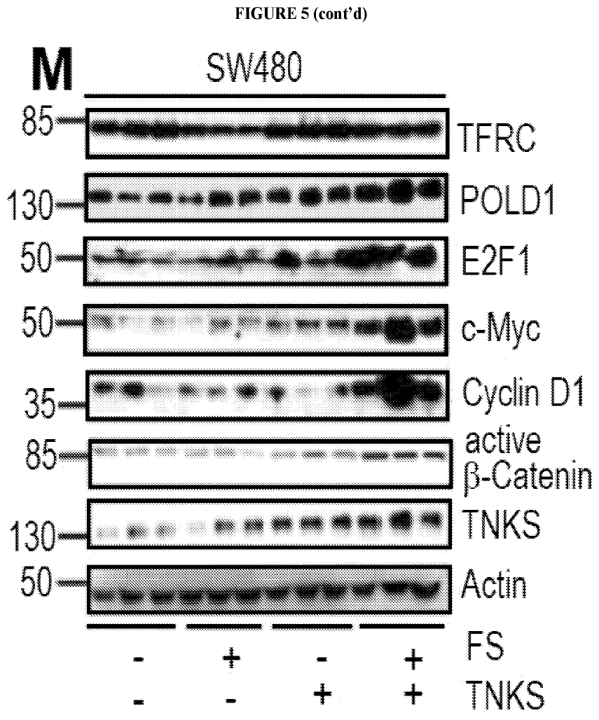

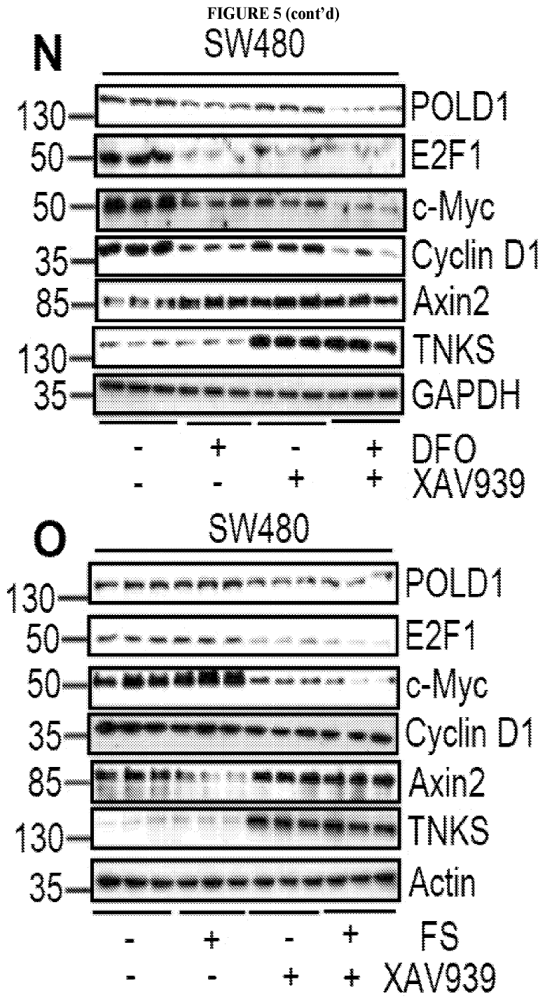

To gain insights on how iron reduction affects colon tumorigenesis, we treated patient-derived tumor colonoids with iron chelator deferoxamine (DFO) and performed an RNA-seq analysis. Using differential gene expression analysis followed by the Database for Annotation, Visualization and Integrated Discovery (DAVID) functional annotation and Kyoto Encyclopedia of Genes and Genomes (KEGG) pathway enrichment²¹, we discovered that the gene expression of two iron-containing proteins involved in nucleotide metabolism, ribonucleotide reductase regulatory subunit M2 (RRM2) and POLD1, were decreased 3.28 and 4.31-fold by DFO in tumor colonoids, respectively (FIG. 5A). RRM2 is regulated by p53 signaling and catalyzes the formation of deoxyribonucleoside diphosphate (dNDP) from nucleoside diphosphates (NDP), whereas POLD1 is a key DNA polymerase that synthesizes DNA from deoxyribonucleoside triphosphate (dNTP)²². By qPCR analysis, we confirmed that DFO treatment significantly reduced the mRNA expression of RRM2 and POLD1 in tumor colonoids (FIG. 5B). The protein expression of POLD1—but not RRM2—was decreased, whereas the DNA damage marker p-p53 was increased by DFO in colon-derived HCT116 and SW480 cells (FIG. 5C). POLD1 was pulled down by ferrous iron beads in our previous study⁸, indicating a direct binding between iron and POLD1. We further confirmed that POLD1 could directly bind to iron in HCT116 cells by immunoblotting analysis (FIG. 5D). Moreover, ferrous sulfate (FS) supplementation can rescue DFO-decreased POLD1 (FIG. 5E). However, iron alone did not induce POLD1 (FIG. 5E). Furthermore, a 2-week low iron diet (3.5 ppm Fe) induced TFRC but decreased POLD1 in mouse colons (FIG. 5F). DFO is known to activate HIF signaling²³, whereas p53 can repress POLD1²⁴. However, neither inhibiting HIF signaling nor p53 knockout rescued DFO-repressed POLD1 (FIGS. S5A-S5C). E2F1 is a POLD1 activating transcription factor and a direct target gene of c-Myc^{25, 26}, whereas c-Myc can be reduced by DFO²⁷. We found that E2F1, c-Myc, POLD1 and FTH1 were reduced, whereas HIF-2α was increased by DFO in a dose-dependent manner as expected (FIG. S5D). Importantly, E2F1 and c-Myc overexpression rescued DFO-repressed POLD1 expression (FIGS. 5G, 5H, S5E, S5F), whereas E2F1 knockdown, c-Myc knockdown and c-Myc inhibitor 10058-F4 reduced E2F1 and POLD1 (FIG. 5I, 5J, S5G). These results indicate that c-Myc and E2F1 play a critical role in POLD1 regulation under iron starvation.

c-Myc is a known β-catenin direct target gene²⁸, whereas Wnt inhibitor screen reveals β-catenin signaling is iron-dependent²⁹. Consistently, we found that iron chelation significantly reduced basal and β-catenin induced Topflash luciferase activity (FIG. S5H). Though iron can amplify Wnt signaling and induce c-Myc expression in SW480 cells with APC mutation³⁰; surprisingly iron didn't activate or potentiate Wnt signaling in our hands⁸. Immunoblotting analysis confirmed that iron reduced TFRC, the iron-sensing protein IRP2 and Axin2, increased FTH1, but c-Myc, E2F1, POLD1 and another β-catenin target gene cyclin D1 were unchanged in SW480 cells (FIG. S5I). Interestingly, DFO decreased cyclin D1 as previously reported³¹, but it induced Axin2 (FIG. S5J). Axin2 is a transcriptional target and a negative regulator of β-catenin signaling³². Consistently, Axin2 knockdown by siRNA induced TFRC, c-Myc, E2F1 and POLD1 (FIG. S5K).

TNKS is a Zn binding protein important in Axin2 poly-ADP-ribosylation, ubiquitination and degradation³³. Neither iron supplementation nor chelation changed TNKS expression (FIG. S5L). However, iron chelation significantly reduced basal and TNKS-induced Topflash luciferase activity (FIG. 5K), whereas iron supplementation only increased TNKS-induced but not basal Topflash luciferase activity (FIG. 5L). Furthermore, the combination of iron and TNKS overexpression, but neither treatment alone, greatly activated the TNKS/Axin2/β-catenin signaling and induced TFRC, POLD1, E2F1, c-Myc and Cyclin D1 (FIG. 5M). Moreover, TNKS activity inhibitor XAV939 increased the expression of TNKS and Axin2 as expected¹⁵, and potentiated the suppressive effect of DFO on c-Myc, E2F1, POLD1 and Cyclin D1 (FIG. 5N). In addition, XAV939 rescued iron-repressed Axin2 and suppressed c-Myc, E2F1, POLD1 and Cyclin D1 even with iron supplementation (FIG. 50). Noteworthy, Axin2 was significantly increased in the colon tumors than in normal colons from 40Fe treated CDX2^{ERT2}Apc^{F/+} mice, but was decreased by 1000 Fe in both normal and tumor colons (FIG. S5N). Collectively, these data indicate that iron is essential for the TNKS activity and subsequent regulation of β-catenin/c-Myc/E2F1/POLD1 signaling.

### Tfrc Depletion Leads to Decreased Iron Levels, POLD1 Expression and Tumor Growth.

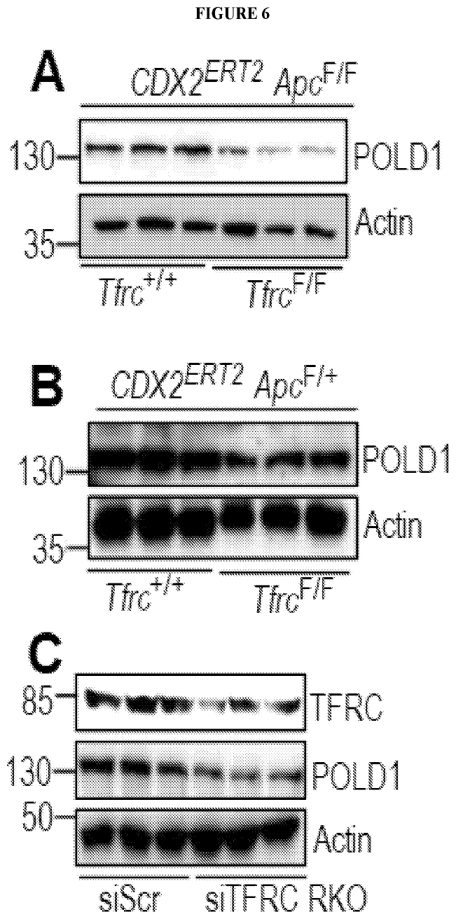

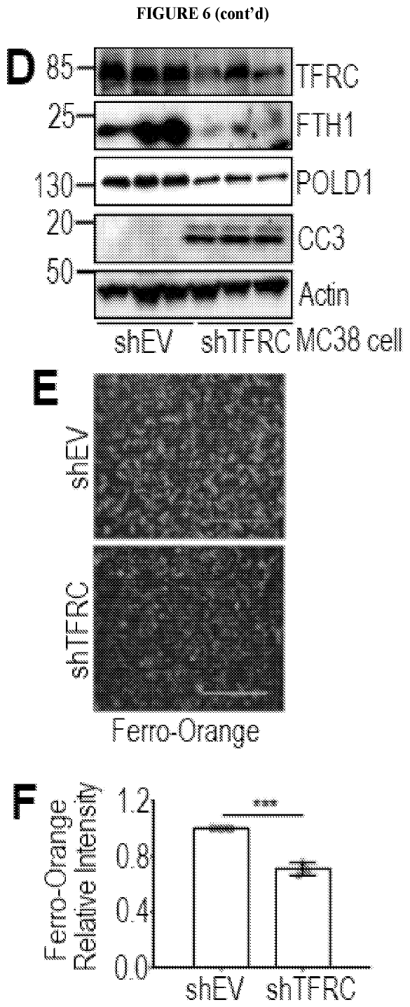

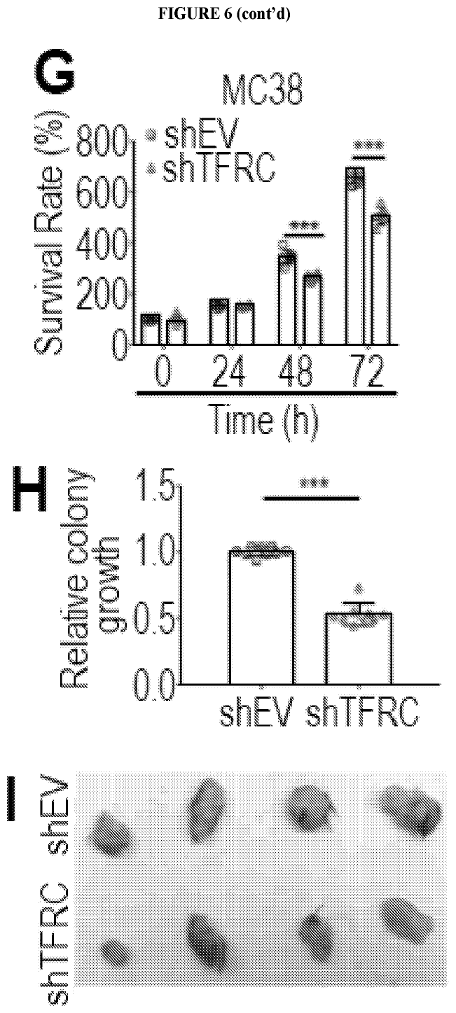

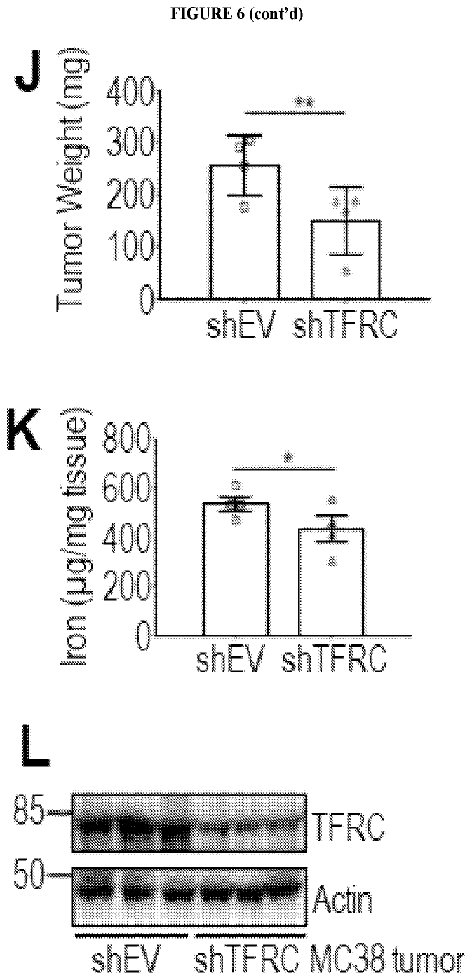

Because TFRC knockout leads to decreased iron in colon and iron starvation can reduce POLD1 expression, we further investigated whether TFRC disruption could reduce POLD1 expression. Indeed, POLD1 was decreased by TFRC disruption (FIG. 6A, 6B). To confirm the TFRC-mediated change of POLD1 is a direct effect in epithelial cells, TFRC was knocked down by siRNA in colon-derived RKO cells. siTFRC simultaneously decreased TFRC and POLD1 (FIG. 6C). Moreover, TFRC, FTH1, POLD1, E2F1 and c-Myc were decreased, whereas CC3 and Axin2 were increased and TNKS was not changed in murine CRC MC38 shTFRC cells (FIG. 6D, S6A). Consistently, the TFRC knockdown significantly decreased Topflash activity (FIG. S6B). Furthermore, E2F1 overexpression rescued TFRC knockdown-mediated POLD1 reduction and CC3 activation (FIG. S6C). To further confirm that TFRC knockdown caused cellular iron status change, we performed Ferro-Orange staining. FS greatly increased, whereas DFO greatly reduced the orange fluorescence signal in MC38 cells (FIG. S6D), indicating that this dye is a good probe for cellular ferrous iron levels. Thus, the decreased fluorescence intensity in MC38 shTFRC cells indicates reduced cellular ferrous iron levels (FIG. 6E, 6F). Both MTT assay and colony formation assay showed that MC38 shTFRC cells had reduced cell growth (FIGS. 6G, 6H, S6E). Furthermore, MC38 shTFRC cell-derived tumor xenografts were smaller (FIG. 6I), and tumor weights were lighter (FIG. 6J). Decreased tumor tissue iron and TFRC were also observed (FIGS. 6K, 6L). Together, our data reveal that TFRC depletion in colon epithelial cells can lead to decreased cellular iron levels, reduced POLD1 expression, increased apoptosis and repressed tumor growth.

### POLD1 Inhibition Causes Increased DNA Replicative Stress and Impaired Tumor Growth.

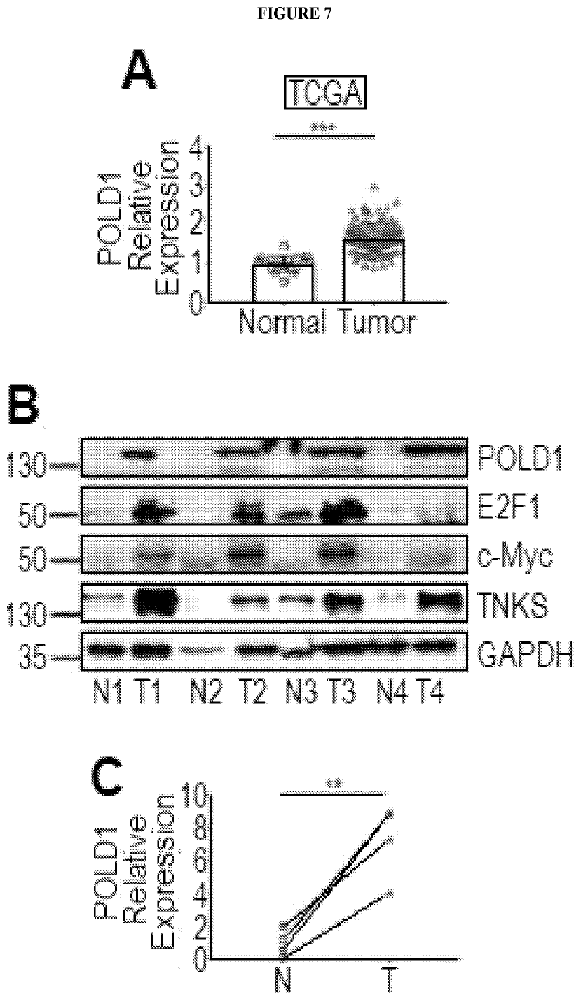

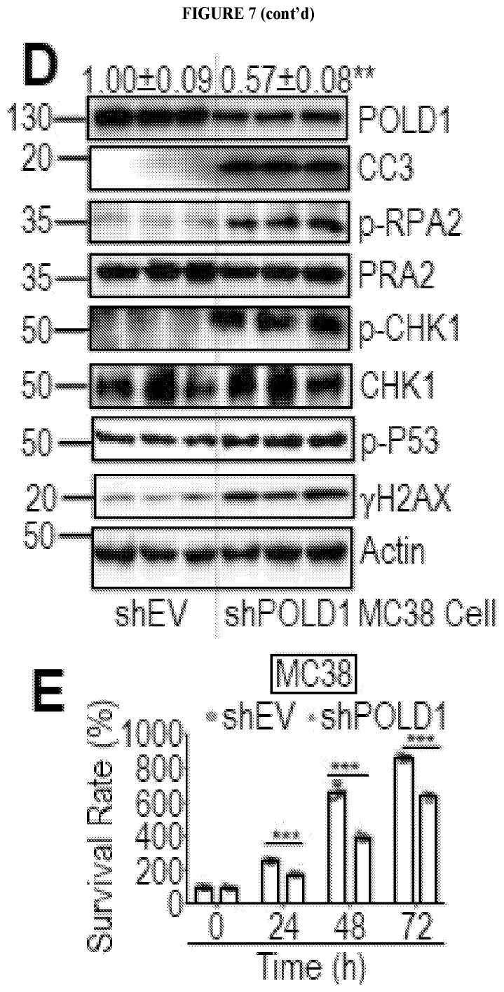

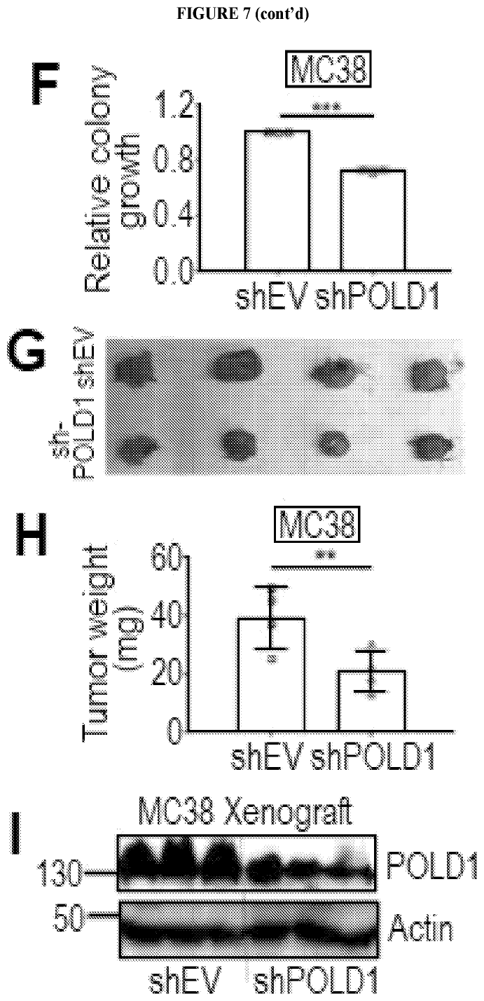

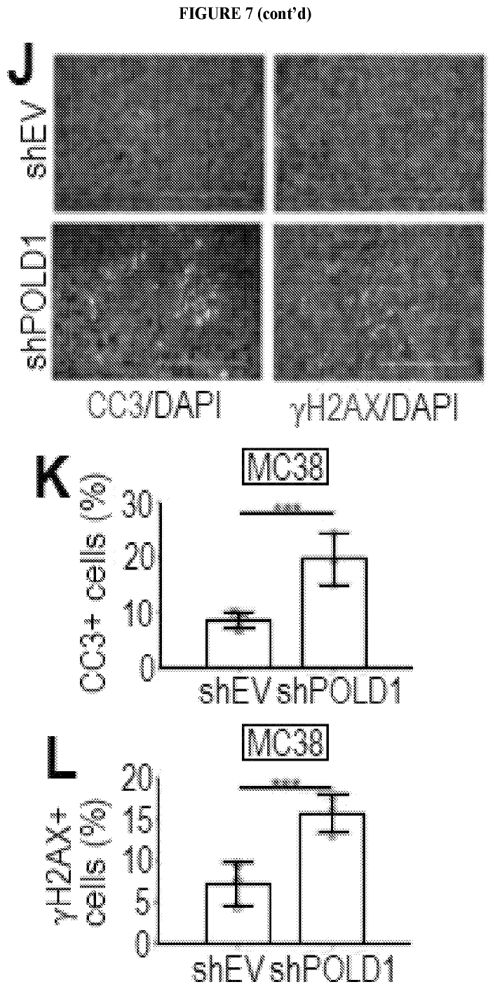

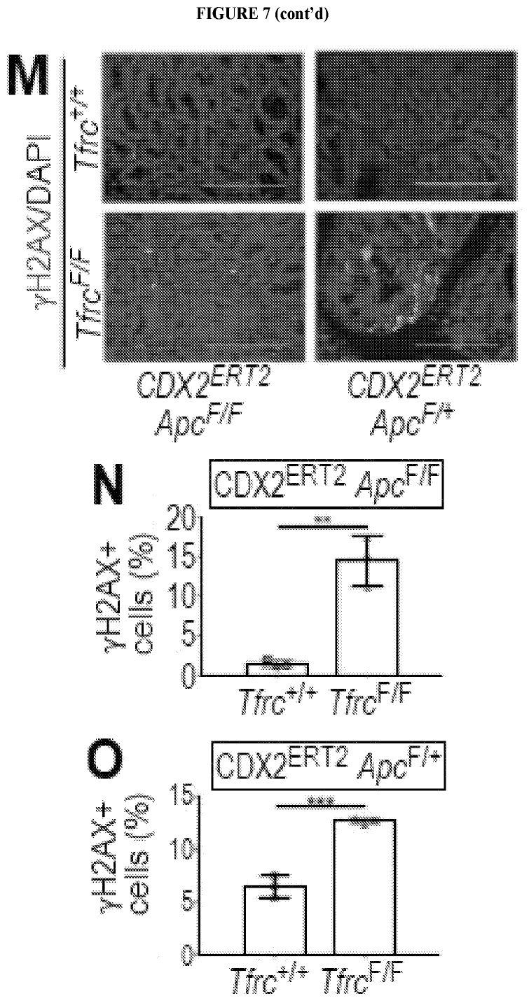

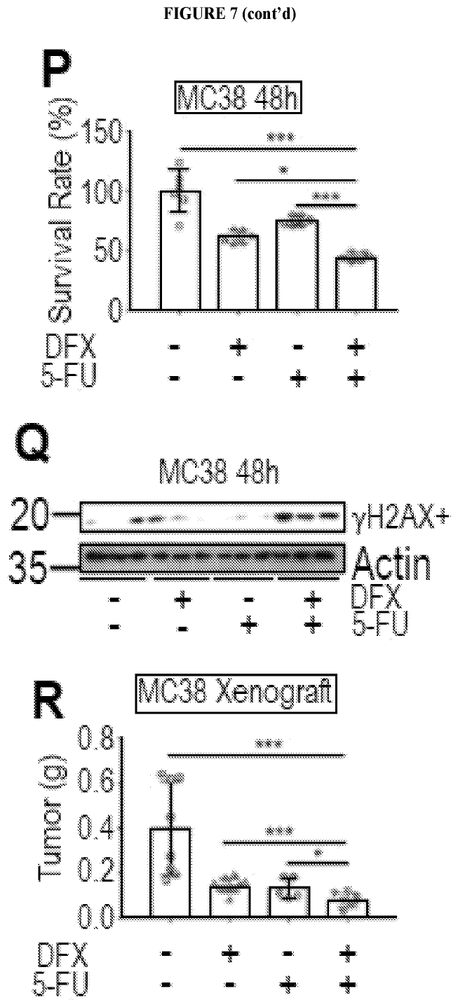

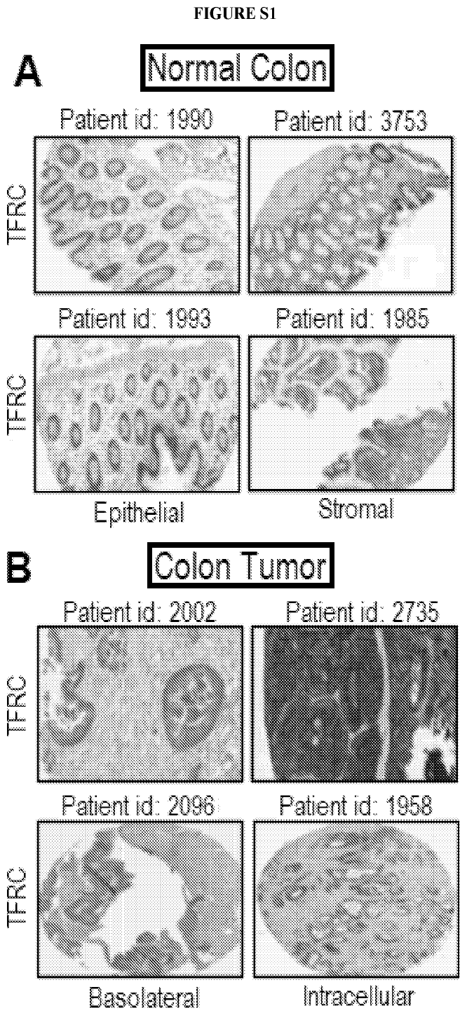

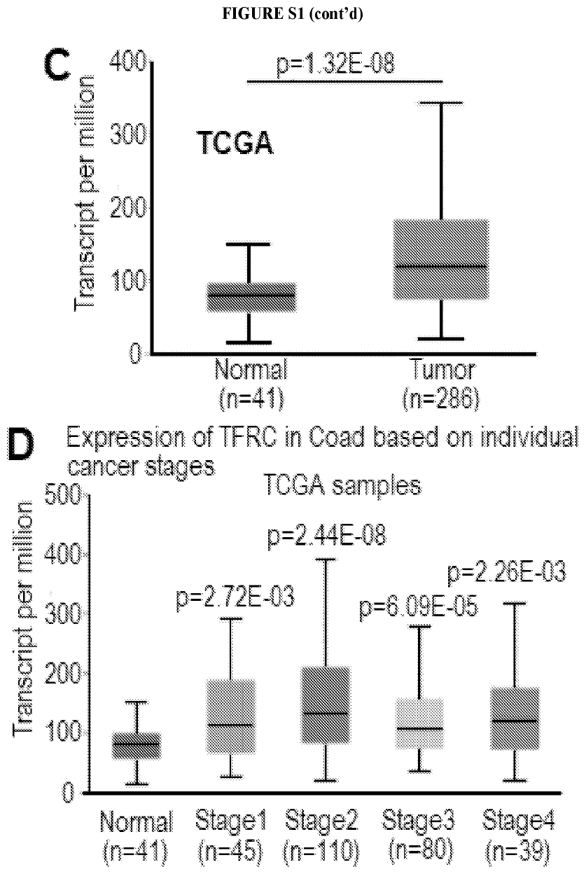

To further investigate the role of POLD1 in colon tumorigenesis, we first examined POLD1 in human CRC tissues. The mRNA expression of POLD1 was significantly increased in tumors compared to normal colons from TCGA database (FIG. 7A). We further confirmed that the protein expression of POLD1 in CRC tumors, together with E2F1, c-Myc and TNKS, was increased in a small cohort of paired patient samples (FIGS. 7B, 7C). Immunohistochemistry staining from HPA showed that POLD1 was highly increased in colon tumors (FIG. S7A). Besides DNA replication, POLD1 has emerged as a pivotal protein in genome maintenance and POLD1 deficiency leads to replicative stress³⁴. DNA replicative stress can occur when oncogenes, genotoxic agents, or inhibitors of DNA replication cause stalled replication forks, leading to activation of DDR pathways. Replicative stress also leads to the phosphorylation of the replication fork component, replication protein A2 (RPA2) to initiate DNA checkpoint signaling³⁵. Checkpoint Kinase 1 (CHK1) is activated by Ataxia Telangiectasia and Rad3-related protein (ATR) phosphorylation to resolve incomplete DNA replication³⁶. Unresolved replicative stress can lead to cell death by replication catastrophe, in which exhaustion of replication factors, such as RPA2, triggers widespread DNA double-strand breaks marked by γ-H2AX³⁷. Importantly, POLD1 knockdown decreased POLD1, resulting in increased p-RPA2, p-CHK1, γH2AX, p-p53 and apoptosis marker CC3 in MC38 cells (FIG. 7D). Consistently, both MTT assay and colony formation assay showed that MC38 shPOLD1 cells had less cell growth (FIG. 7E, 7F). These results were reproducible in RKO cells with POLD1 knockdown (FIGS. S7B, S7C). Furthermore, MC38 shPOLD1 cell-derived tumor xenografts were smaller (FIG. 7G), and tumor weights were lighter (FIG. 7H). Immunoblotting confirmed that POLD1 levels were still reduced in xenograft tumors from shPOLD1 cells than controls (FIG. 7I). IF staining showed that CC3 and γH2AX were significantly increased in xenograft tumors from shPOLD1 cells than controls (FIG. 7J-7L). Noteworthy, γH2AX was also increased in colon dysplastic tissues from CDX2^{ERT2} Tftc^{F/F} Apc^{F/F} mice and colon tumors from CDX2^{ERT2} Tfrc^{F/F} Apc^{F/+} mice (FIGS. 7M-7O). Consistently, DFO treatment dose-dependently increased p-RPA2, p-CHK1, p-p53, γH2AX and CC3 in CRC cells (FIG. S7D).

A recent synthetic lethal screen showed that ATR- or CHK1-inhibitors potentiates caspase-dependent apoptosis in POLD1-deficient cancers³⁸. Another study showed that pharmacologic blockade of B-family DNA polymerases using aphidicolin combined with CHK1 inhibitors also led to synergistic inhibition of cancer cell proliferation³⁹. However, aphidicolin is too toxic to be used in humans and currently no other suitable drug candidates that can block the DNA polymerase family exist. We further tested the combinatorial effect of DFO and a potent CHK1 inhibitor UCN-01 at low doses on the survival of CRC cells. We found that 10 μM DFO and 100 nM UCN-01 synergistically inhibited cell growth in both SW480 and RKO cells (FIG. S7E). Ibis synergistic effect was further validated with a more specific CHK1 inhibitor Prexasertib⁴⁰ (FIG. S7F), an oral iron chelator deferasirox (DFX) (FIG. S7G) and an ATR inhibitor VE-822 (FIG. S7H). Interestingly, the combination of DFX with Prexasertib or VE-822 synergistically decreased POLD1 and increased γH2AX (FIGS. S7I, S7J), which was used as a bioactivity marker for CHK1 inhibitors⁴¹. Importantly, 5-FU, a standard first-line DNA damaging chemotherapy drug for CRC, also potentiated the growth suppressive effect and DDR of DFX on MC38 cells in vitro and in vivo (FIG. 7P-7R). Collectively, we propose that the synergistic effect between iron chelation and inhibition of DDR may be utilized as a novel therapeutic strategy for CRC.

### Discussion

Using a novel colon-specific TFRC knockout mouse model, we demonstrated that TFRC disruption caused mild colon injury shown as increased epithelial cell apoptosis and enhanced susceptibility to acute colitis under low-iron diet treatment. These data are consistent with the concept that low-iron condition induces TFRC to increase iron for cell survival. In contrast, excess iron is expected to decrease TFRC to reduce iron uptake and toxicity. However, we found that TFRC expression was paradoxically induced in iron-enriched colon tumors and further potentiated by a high-iron diet. TFRC was significantly induced in the colons after Apc gene disruption, whereas TFRC disruption impeded colon tumorigenesis resulting from Apc loss by decreasing intratumoral iron accumulation. Mechanistic study revealed that abundant TFRC-mediated iron uptake in colon tumors is required for maintaining the activity of the metal-dependent TNKS, which caused poly-ADP-ribosylation and degradation of Axin2, and activation of β-catenin/c-Myc/E2F1/POLD1 signaling (FIG. S8A). TFRC deficiency-elicited low iron decreased TNKS activity, stabilized Axin2, increased β-catenin phosphorylation and degradation, suppressed c-Myc/E2F1/POLD1 transcription, and increased DNA replicative stress, DNA damage and cell apoptosis (FIG. S8B). Axin2 knockdown increased, whereas TNKS knockdown decreased the expression of TFRC. Furthermore, overexpression of TNKS potentiated β-catenin signaling in the presence of iron and rescued iron-repressed TFRC. Thus, we hypothesize that TFRC is induced by active β-catenin signaling, whereas TFRC-mediated intratumoral iron accumulation potentiates β-catenin signaling via directly enhancing the activity of TNKS (FIG. S8C). TFRC-mediated iron uptake is at the center of this feed-forward loop to facilitate tumor cell survival in CRC.

It is intriguing that iron suppressed Axin2 expression, but failed to induce c-Myc/E2F1/POLD1 axis. However, Axin2 knockdown and TNKS overexpression with iron supplementation activated c-Myc/E2F1/POLD1 axis. c-Myc is greatly induced by biallelic Apc inactivation in mouse intestines⁴², which is completely abolished by monoallelic inactivation of β-catenin encoding gene Ctnnb1⁴³. The TNKS/c-Myc/E2F1/POLD1 axis was also increased in human colon tumors. Thus, it is important to determine the mechanism whereby drives TNKS overexpression in CRC in the future.

POLD1 is an iron-sulfur cluster containing protein and the iron-sulfur cluster was incorporated by the cytosolic iron-sulfur cluster assembly system⁴⁴. Thus, iron is known to modulate the stability and activity of POLD1 at post-transcriptional level. Our data further showed that reduced iron can also repress POLD1 at transcriptional level through TNKS/Axin2/β-catenin/c-Myc/E2F1 signaling. Germline and non-silent somatic mutations in the exonuclease proofreading domains of POLD1 predisposes patients to develop hypermutated sporadic and colitis-associated CRC²². We found POLD1 was correlated with c-Myc. Thus, c-Myc is the rheostat that connects POLD1 expression with β-catenin signaling in CRC.

Due to the fact that TFRC is overexpressed in a variety of human tumors, antibodies targeting TFRC are attractive and straightforward therapeutic options¹⁴. However, the safety for this type of treatment is a big concern, because many normal tissues like bone marrow and lymphoid tissues express high levels of TFRC. In contrast, the FDA approved iron chelator DFO has a relatively safe record in the clinical treatment of iron overload diseases. DFO treatment resulted in 20% overall response rate in a cohort of 10 advanced hepatocellular carcinoma without severe adverse events⁴⁵. However, DFO's extremely short circulation half-life restricts its use as an effective antitumor agent⁴⁶. When DFO is used in iron overload diseases, the drug must be administered by continuous subcutaneous infusion for up to 12 h per day for 5-7 days per week, which leads to suboptimal adherence to therapy by patients⁴⁷. The low ability of DFO to cross cell membranes also limits its efficacy⁴⁸. Two next generation orally administered iron chelators deferiprone and DFX are now in routine clinical use for treating iron overload diseases, but these agents have not been used as extensively as DFO due to their acute toxicities⁴⁹.

To reduce potential acute toxicities and maintain its efficacy, DFX has been investigated for combinatorial therapy in breast cancer¹⁴. However, DFX or other iron chelators-based drug combination for CRC treatment has not been reported yet. A metal chelator, tachpyridine, induces G2 cell cycle arrest via ATR-dependent activation of CHK1, and sensitizes cancer cells (not noncancer cells) to ionizing radiation induced DNA damage⁵⁰. DNA damage signaling inhibitors mainly target kinases (e.g., ATR, CHK1) that phosphorylate a range of proteins involved in triggering cell-cycle arrest to enable DNA repair⁵¹. Since FDA approved the first poly-ADP-ribose polymerase inhibitor olaparib in 2014, the development of novel DDR targets has gained enormous attention⁵². However, DDR inhibitor monotherapy often has limited efficacy for cancer treatment⁵³. Here we have shown that the combinatorial inhibition of POLD1 by iron chelation and DDR by DNA damaging agents had a synergistic effect on inducing DNA damage and suppressing CRC cell growth (FIG. S8C).

In summary, TFRC-mediated iron uptake is essential for colon homeostasis via modulating TNKS/Axin2/β-catenin/c-Myc/E2F1/POLD1 axis. An iron chelation-based chemotherapy strategy exhibits activity consistent with its use in cancer chemotherapy.

### Additional Experiments

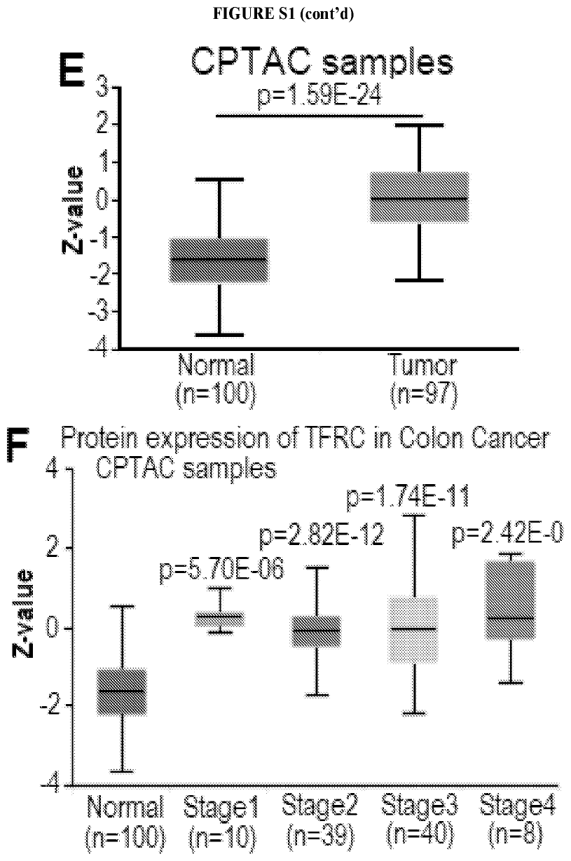

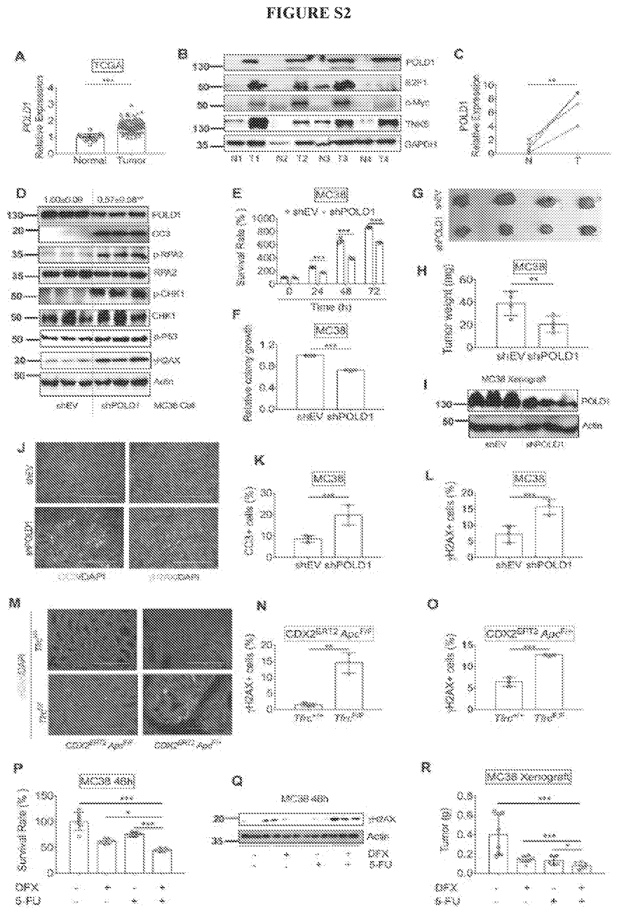

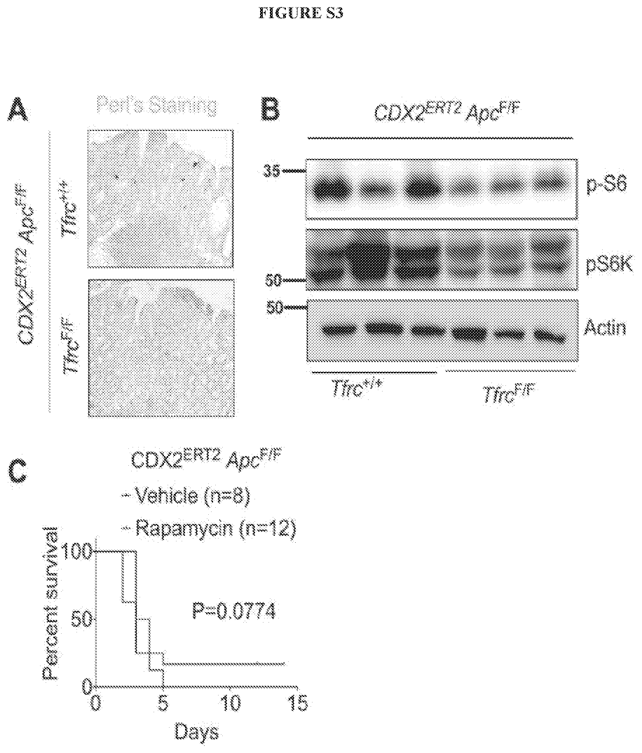

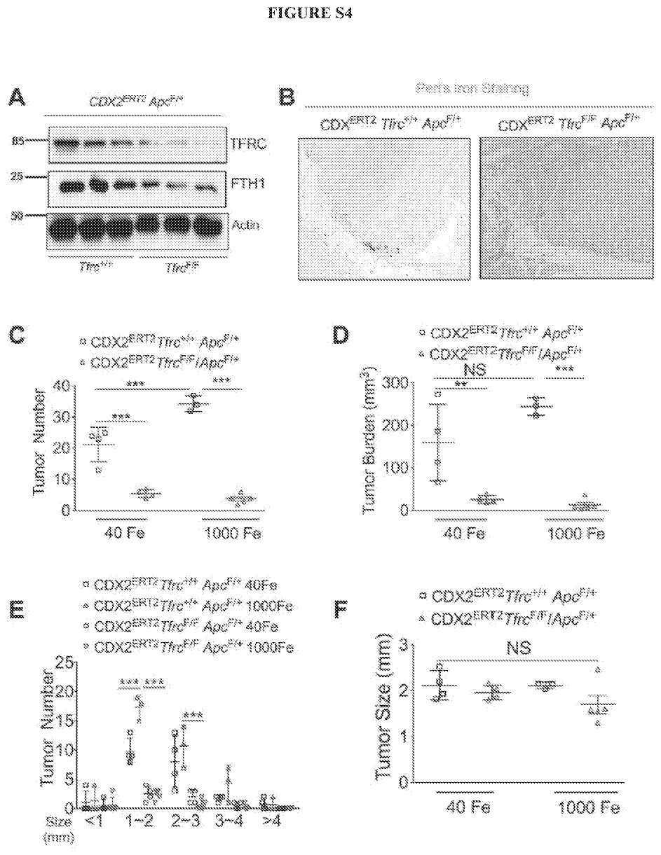

The fact that iron starvation does not alter normal colon proliferation, suggests a selectivity of iron for cancer cells and provides an ideal target for CRC therapy. Of all the iron chelators used clinically, DFO has been used the longest, but the drug's extremely short circulation half-life of approximately 20 min in humans, and 5 min in mice, restricts its use as an antitumor agent 89, 90. Two next generation orally administered iron chelators deferiprone and deferasirox (DFX) are now in routine clinical use, but these agents have not been used as extensively as DFO due to their acute toxicities 91. Synergism between compounds has been an area of interest for researchers as a combinatorial approach can improve efficacy and mitigate toxicity. It was reported that DFO-induced apoptosis occurs through the ataxia-telangiectasia-Rad3-related kinase (ATR)-checkpoint kinase CHK1 DNA damage signaling pathway 92. Consistently, we found that DFO treatment dose-dependently increased DNA damage signaling markers p-CHK1 Ser317, p-P53 Ser15 and γH2AX, replicative stress marker RPA-2 and apoptosis marker CC3 in HCT116 and SW480 cells (FIG. 8A). Importantly, γH2AX was also increased in colon dysplastic tissues from CDX2ERT2 TfrcF/FApcF/F mice and colon tumor tissues from CDX2ERT2 TfrcF/FApcF/+ mice (FIG. 8B). These results indicated that iron restriction led to activation of DNA damage response.

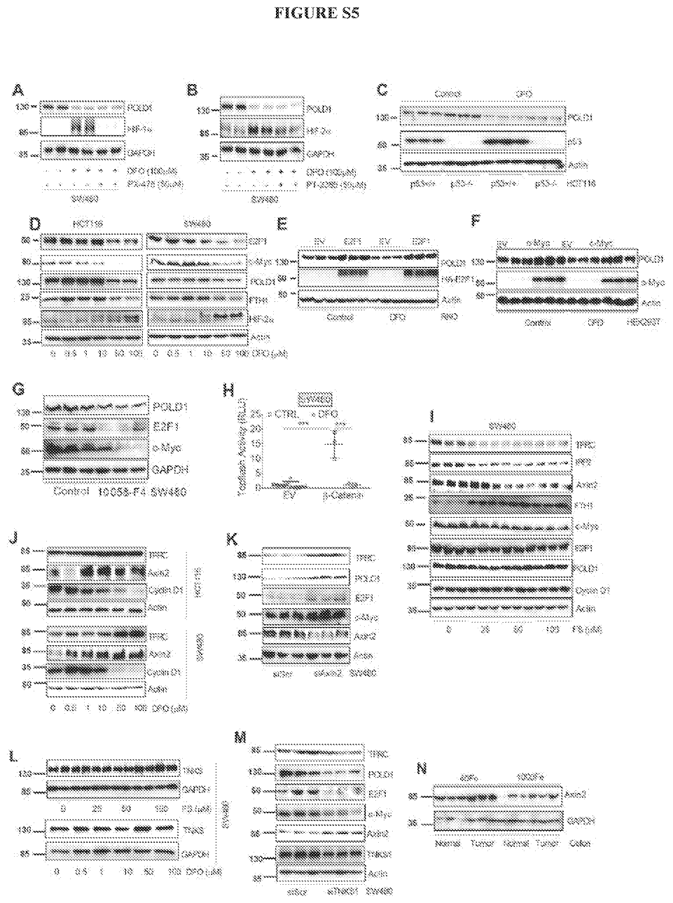

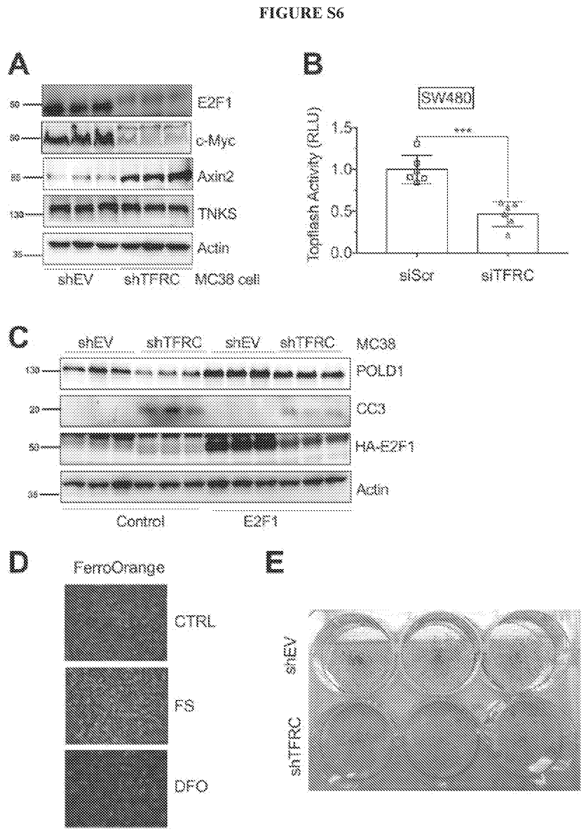

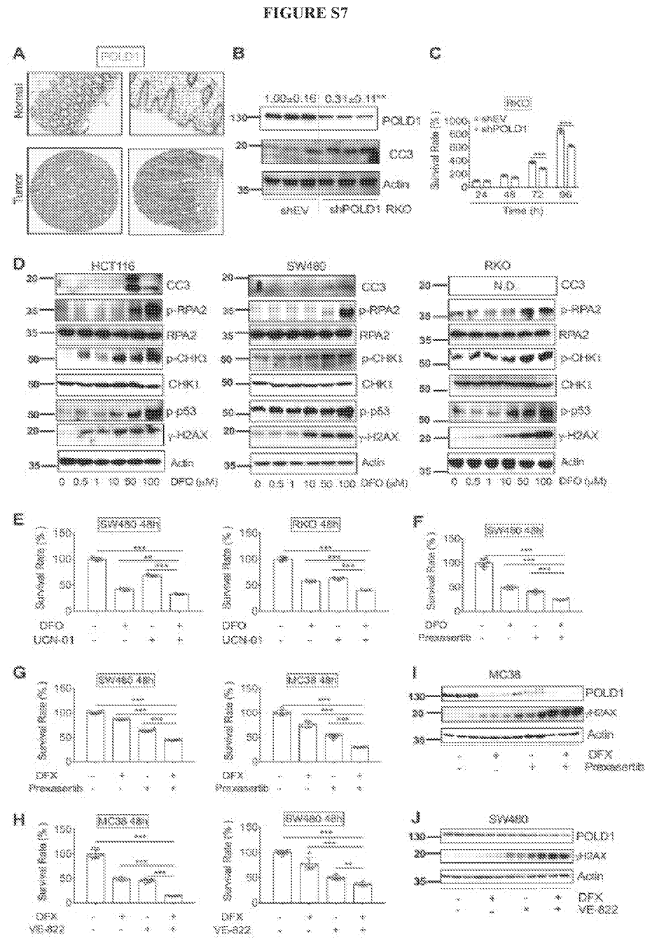

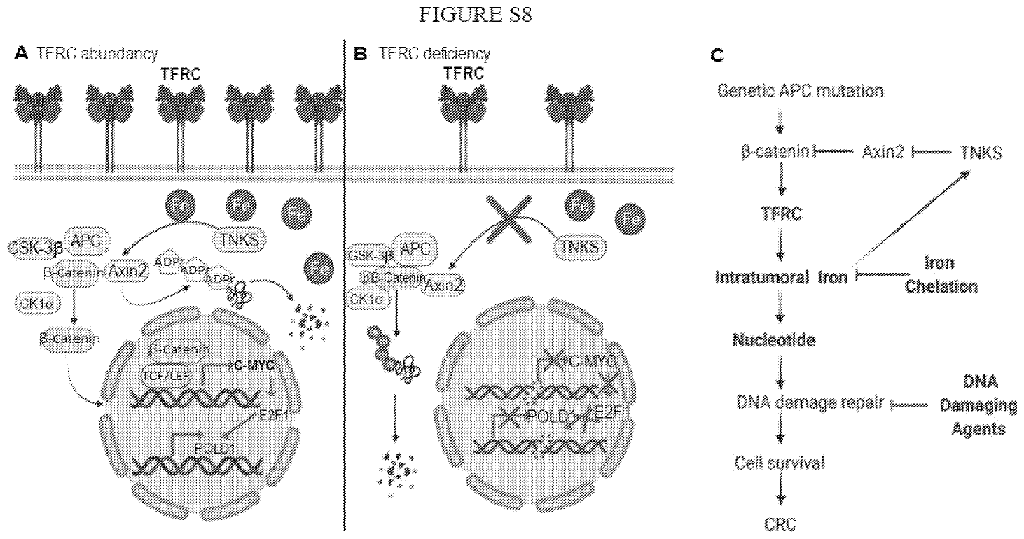

A previous report showed that a metal chelator, tachpyridine, induces G2 cell cycle arrest via ATR-dependent activation of CHK1, and sensitizes cancer cells (not noncancer cells) to ionizing radiation induced DNA damage. DNA damage signaling inhibitors mainly target kinases (e.g., ATR, ATM, CHK1, WEE1) that phosphorylate a range of proteins involved in triggering cell-cycle arrest to enable DNA repair. Iron chelation reduced the abundance of nucleotides, which are the building blocks for repairing damaged DNA. Thus, the inventor tested the combinational effects of DFX and a specific CHK1 inhibitor Prexasertib on the survival in CRC cells. It was found that low dose of DFX (10 μM) promoted Prexasertib inhibited cell growth in SW480 cells (FIG. 9A) and MC38 cells (FIG. 9B). Consistently, DFX potentiated the level of Prexasertib-induced γH2AX, which was used as a bioactivity marker for CHK1 inhibitors (FIG. 9C, 9D). The combinations of DFX with an ATR inhibitor VE-822 (FIG. 9E, 9F), DFO with another potent CHK1 inhibitor UCN-01 (data not shown), had similar effects.

The inventor designed a series of experiments to test the hypothesis that iron chelation may be exploited as a combinatorial therapy with DDR modulators for CRC treatment. These studies will determine the combinational effect in vitro and in vivo. To my knowledge, this will be one of the few studies to systemically assess iron chelator-based combinatorial treatments for CRC.

### Characterization of the Best Combination of Iron Chelation and Genotoxic Chemotherapy for Killing CRC Cells.

Genotoxic chemotherapy increases pyrimidine nucleotide levels, whereas pharmacologic inhibition of de novo pyrimidine synthesis sensitizes triple-negative breast cancer cells to genotoxic chemotherapy agents by exacerbating DNA damage. Iron chelation reduces de novo pyrimidine synthesis. Here we perform a small drug screen to determine the best combination and quantify synergy between iron chelators and genotoxic chemotherapy agents in four human CRC cell lines (HCT116, SW480, RKO and DLD-1) with or without APC mutation using an MTT proliferation assay. Iron chelators include three FDA-approved drugs (DFO, deferiprone and DFX) as well as two clinically-investigated thiosemicarbazones (Triapine and Dp44mT). The genotoxic chemotherapy agents will include standard chemotherapy drugs used to treat CRC (5-FU, Oxaliplatin, Irinotecan), a poly-ADP-ribose polymerase inhibitor Olaparib, an intercalating agent Doxorubicin, and two alkylating agents (Cyclophosphamide, Temozolomide). Paclitaxel is used as a non-genotoxic drug control. Three ATR inhibitors (VE-822, BAY1895344 and AZ6738) and two CHK1 inhibitors (Prexasertib and SRA737) that are under active clinical trials are also be tested in combination with iron chelators. Interestingly, none of the above drug combinations have been investigated for CRC treatment, which makes the present invention the first.

MTT proliferation assay: CRC cells are seeded in 96-well plates at a density of 5×103 cells/well in 100 μL of complete DMEM. The cells are allowed to adhere overnight, and the medium is then removed and replaced with fresh medium containing the appropriate concentrations of either the drugs alone or in appropriate combinations. The drug concentrations for combination treatments are based on the initial IC50 values (⅛-, ¼-, ½-, 1-, 2-, 4-, and 8-fold of IC50) of each drug, and therefore, they are specific for each cell line examined. The plates are incubated for 3 days at 37° C.

Calculation of combination index (CI): CIs are used to quantitatively compare the dose-effect relationship of each drug alone or in combination to determine whether a given combination acts synergistically. CIs will be calculated from growth inhibition curves, as previously described. A 1:1 ratio of drugs will be used for combination treatments. The CI values will be determined using free ComboSyn software. The Chou-Talalay method is adopted to identify synergism (CI<0.9), additive effect (CI=0.9-1.1) or antagonism (CI>1.1).

### Assessment of Nucleoside Supplementation to Rescue Iron Chelation-Mediated DNA Damage in CRC.

The inventor has shown supplementation with a cocktail of all four nucleosides partially rescued DFO-mediated growth inhibition in CRC-derived cell lines (Nat Metab, Accepted). Here it is to be determined if oxidative stress and DNA damage are rescued by nucleosides or not. Four human CRC cell lines (HCT116, SW480, RKO and DLD-1) as well as four patient-derived colonoids (569, 584, 781, and 861) are co-treated with a cocktail of all four nucleosides (100 μM adenosine, guanosine, cytidine and thymidine) and DFO (10 μM) for 72 hours. DFO induces oxidative stress by decreasing intracellular glutathione levels and increasing ROS levels 104, 105. Thus, H2DCFDA staining will be used to detect total ROS levels as we have previously described 55. Immunoblots will be used to determine the levels of replication stress marker p-RPA32, and DNA damage signaling markers including p-CHK1 Ser317, γH2AX and p-P53 Ser15.

### Evaluation of the Combinatorial Effect of Iron Chelation-Based Chemotherapy in a Mouse Model of CRC.

The inventor has observed the potentiation effect of DFX and VE-822 in vitro and these have been extensively investigated for other tumors in vivo. Thus, pursuant to these experiments, these test their combinational effects in mouse models of CRC in addition to selected combination as set forth above and as otherwise described herein.

Xenograft study: 1×106 murine MC38 cells are subcutaneously injected into C57BL/6 mice as we have described previously 33, 37. When tumors are palpable (a week later), these mice are treated with vehicle (30% 1,2-propanediol/70% sterile 0.9% sodium chloride solution [v/v]), DFX (orally on alternate days at 20 mg/kg), VE-822 (orally at 60 mg/kg for 6 consecutive days), or both DFX and VE-822. A week later, mice are anaesthetized and exsanguinated by direct cardiac puncture, and blood/plasma is retained for full blood count and biochemical analysis (urea, creatinine, alanine transaminase, aspartate transaminase, albumin, total bilirubin, serum iron and total iron-binding capacity) to assess potential toxicity. The liver, heart and spleen are removed and weighed before immediate division into tubes containing formalin for histological analysis. Tumors are removed and weighed to assess tumor burden. Tumor iron content is determined by Perl's iron staining and ICP-MS, whereas tumor proliferation (Ki67), apoptosis (CC3) and DNA damage (γH2AX) is assessed by IF staining. Iron metabolic gene and protein levels including TFRC, FTH1 and FPN are determined by qPCR and immunoblot analysis.

Data interpretation and alternatives: Iron addiction is not observed in normal tissues, but appears to be specifically increased in colon tumors. This provides a level of specificity that other current or proposed therapies do not address and making it an ideal target for CRC therapy. Chemical inhibition of the key DDR proteins and pharmacologically induced synthetic lethality have become promising anticancer agents. In further experiments, we use an unbiased approach for the first time to determine the best combination of iron chelation and genotoxic chemotherapy in vitro. These experiments comprehensively determine whether the nucleotide pool deficiency is responsible for iron chelation induced DDR. This is the first study in vivo to test the combinatorial effect of iron chelation and DDR modulation in CRC. The doses for DFX and VE-822 in mice were based on the literature. We further test if sequential iron chelation and radiotherapy leads to synthetic lethality in CRC cells, as radiotherapy is routinely used in CRC treatment and causes DNA damage.

### REFERENCES

1. Siegel R L, Miller K D, Jemal A. Cancer statistics, 2020. CA Cancer J Clin 2020; 70: 7-30.
2. DeBerardinis, R.J., Mancuso, A., Daikhin, E., Nissim, I., Yudkoff, M., Wehrli, S., and Thompson, C. B. (2007). Beyond aerobic glycolysis: transformed cells can engage in glutamine metabolism that exceeds the requirement for protein and nucleotide synthesis. Proc Natl Acad Sci USA 104, 19345-19350.
3. Kang, Y.P., Ward, N.P., and DeNicola, G.M. (2018). Recent advances in cancer metabolism: a technological perspective. Exp Mol Med 50, 31.
4. Nelson, R. L. (2001). Iron and colorectal cancer risk: human studies. Nutr Rev 59, 140-148.
5. Cross, A.J., Ferrucci, L. M., Risch, A., Graubard, B. I., Ward, M. H., Park, Y., Hollenbeck, A. R., Schatzkin, A., and Sinha, R. (2010). A large prospective study of meat consumption and colorectal cancer risk: an investigation of potential mechanisms underlying this association. Cancer Res 70, 2406-2414.
6. Merk, K., Mattsson, B., Mattsson, A., Holm, G., Gullbring, B., and Bjorkholm, M. (1990). The incidence of cancer among blood donors. Int J Epidemiol 19, 505-509.
7. Osborne, N. J., Gurrin, L. C., Allen, K. J., Constantine, C. C., Delatycki, M. B., McLaren, C. E., Gertig, D. M., Anderson, G. J., Southey, M. C., Olynyk, J. K., et al. (2010). HFE C282Y homozygotes are at increased risk of breast and colorectal cancer. Hepatology 51, 1311-1318.
8. Zacharski, L. R., at al., Decreased cancer risk after iron reduction in patients with peripheral arterial disease: results from a randomized trial. J Natl Cancer Inst, 2008. 100(14): p. 996-1002.
9. Brookes, M. J., et al., Modulation of iron transport proteins in human colorectal caroinogenesis. Gut, 2006. 55(10): p. 1449-60.
10. Xue, X., et al., Endothelial PAS domain protein 1 activates the inflammatory response in the intestinal epithelium to promote colitis in mice. Gastroenterology, 2013. 145(4): p. 831-41.
11. Xue, X., et al., Iron Uptake via DMT1 Integrates Cell Cycle with JAK-STAT3 Signaling to Promote Colorectal Tumorigenesis. Cell Metab, 2016. 24(3): p. 447-461.
12. Hentze, M. W., M. U. Muckenthaler, and N. C. Andrews, Balancing acts: molecular control of mammalian iron metabolism. Cell, 2004. 117(3): p. 285-97.
13. Parmley, R. T., J. C. Barton, and M. E. Conrad, Ultrastructural localization of transferrin, transferrin receptor, and iron-binding sites on human placental and duodenal microvilli. Br J Haematol, 1985. 60(1): p. 81-9.
14. Anderson, G. J., L. W. Powell, and J. W. Halliday, Transferrin receptor distribution and regulation in the rat small intestine. Effect of iron stores and erythropoiesis. Gastroenterology, 1990. 98(3): p. 576-85.
15. Banerjee, D., et al., Transferrin receptors in the human gastrointestinal tract. Relationship to body iron stores. Gastroenterology, 1986. 91(4): p. 861-9.
16. Chen, A. C., et al., Noncanonical role of transferrin receptor 1 is essential for intestinal homeostasis. Proc Natl Acad Sci USA, 2015. 112(37): p. 11714-9.
17. Shen, Y., et al., Transferrin receptor 1 in cancer: a new sight for cancer therapy. Am J Cancer Res, 2018. 8(6): p. 916-931.
18. Moura I C, Lepelletier Y, Arnulf B, England P, Baude C, Beaumont C et al. A neutralizing monoclonal antibody (mAb A24) directed against the transferrin receptor induces apoptosis of tumor T lymphocytes from ATL patients. Blood 2004; 103: 1838-1845.
19. Daniels, T. R., et al., The transferrin receptor part I: Biology and targeting with cytotoxic antibodies for the treatment of cancer. Clin Immunol, 2006. 121(2): p. 144-58.
20. Ng P P, Dela Cruz J S, Sorour D N, Stinebaugh J M, Shin S-U, Shin D S et al. An anti-transferrin receptor-avidin fusion protein exhibits both strong proapoptotic activity and the ability to deliver various molecules into cancer cells. Proc Natl Aced Sci USA 2002; 99: 10706-10711.
21. Ng P P, Helguera G, Daniels T R, Lomas S Z, Rodriguez J A, Schiller G et al. Molecular events contributing to cell death in malignant human hematopoietic cells elicited by an IgG3-avidin fusion protein targeting the transferrin receptor. Blood 2006; 108: 2745-2754.
22. Daniels-Wells T R, Candelaria P V, Leoh L S, Nava M, Martinez-Maza O, Penichet M L. An IgG1 Version of the Anti-transferrin Receptor 1 Antibody ch128.1 Shows Significant Antitumor Activity Against Different Xenograft Models of Multiple Myeloma: A Brief Communication. J Immunother 2020; 43.
23. Cui, C., et al., Downregulation of TfR1 promotes progression of colorectal cancer via the JAK/STAT pathway. Cancer Manag Res, 2019. 11: p. 6323-6341.
24. Uhlén M, Fagerberg L, Hallström B M, Lindskog C, Oksvold P, Mardinoglu A et al. Tissue-based map of the human proteome. Science (80-) 2015; 347: 1260419.
25. Wilkinson, N. and K. Pantopoulos, The IRP/IRE system in vivo: insights from mouse models. Front Pharmacol, 2014. 5: p. 176.
26. Hinoi T, Akyol A, Theisen B K, Ferguson D O, Greenson J K, Williams B O et al. Mouse Model of Colonic Adenoma-Carcinoma Progression Based on Somatic Apc Inactivation. Cancer Res 2007; 67: 9721 LP-9730.
27. Goss, K. H. and J. Groden, Biology of the adenomatous polyposis coli tumor suppressor. J Clin Oncol, 2000. 18(9): p. 1967-79.
28. Watson A, Lipina C, McArdle H J, Taylor P M, Hundal H S. Iron depletion suppresses mTORC1-directed signalling in intestinal Caco-2 cells via induction of REDD1. Cell Signal 2016; 28: 412-424.
29. Faller W J, Jackson T J, Knight J R, Ridgway R A, Jamieson T, Karim S A et al. mTORC1-mediated translational elongation limits intestinal tumour initiation and growth. Nature 2015; 517: 497-500.
30. Huang D W, Sherman B T, Tan Q, Kir J, Liu D, Bryant D et al. DAVID Bioinformatics Resources: expanded annotation database and novel algorithms to better extract biology from large gene lists. Nucleic Acids Res 2007; 35: W169-WI75.
31. Wang, F., Zhao, Q., Wang, Y.-N., Jin, Y., He, M.-M., Liu, Z.-X., and Xu, R.-H. (2019). Evaluation of POLE and POLD1 Mutations as Biomarkers for Immunotherapy Outcomes Across Multiple Cancer Types. JAMA Oncol. 5, 1504-1506.
32. Palles, C., Cazier, J.-B., Howarth, K. M., Domingo, E., Jones, A. M., Broderick, P., Kemp, Z., Spain, S. L., Guarino, E., Salguero, I., et al. (2013). Germline mutations affecting the proofreading domains of POLE and POLD1 predispose to colorectal adenomas and carcinomas. Nat. Genet. 45, 136-144.
33. Wang G L, Semenza G L. Desferrioxamine induces erythropoietin gene expression and hypoxia-inducible factor 1 DNA-binding activity: implications for models of hypoxia signal transduction. Blood 1993; 82: 3610-3615.
34. Li, B. and M. Y. Lee, Transcriptional regulation of the human DNA polymerase delta catalytic subunit gene POLD1 by p53 tumor suppressor and Sp1. J Biol Chem, 2001. 276(32): p. 29729-39.
35. Gao S, Song Q, Liu J, Zhang X, Ji X, Wang P. E2F1 mediates the downregulation of POLD1 in replicative senescence. Cell Mol Life Sci 2019; 76: 2833-2850.
36. O'Donnell K A, Wentzel E A, Zeller K I, Dang C V, Mendell J T. c-Myc-regulated microRNAs modulate E2F1 expression. Nature 2005; 435: 839-843.
37. Leung J Y, Ehmann G L, Giangrande P H, Nevins J R. A role for Myc in facilitating transcription activation by E2F1. Oncogene 2008; 27: 4172-4179.
38. Kyriakou D, Eliopoulos A G, Papadakis A, Alexandrakis M, Eliopoulos G D. Decreased expression of c-myc oncoprotein by peripheral blood mononuclear cells in thalassaemia patients receiving desferioxamine. Eur J Haematol 1998; 60: 21-27.
39. Hocke S, Guo Y, Job A, Orth M, Ziesch A, Lauber K et al. A synthetic lethal screen identifies ATR-inhibition as a novel therapeutic approach for POLD1-deficient cancers. Oncotarget 2016; 7: 7080-7095.
40. Rogers R F, Walton M I, Cherry D L, Collins I, Clarke P A, Garrett M D et al. CHK1 Inhibition Is Synthetically Lethal with Loss of B-Family DNA Polymerase Function in Human Lung and Colorectal Cancer Cells. Cancer Res 2020; 80: 1735 LP—1747.
41. Lee J-M, Nair J, Zimmer A, Lipkowitz S, Annunziata C M, Merino M J et al. Prexasertib, a cell cycle checkpoint kinase 1 and 2 inhibitor, in BRCA wild-type recurrent high-grade serous ovarian cancer a first-in-class proof-of-concept phase 2 study. Lancet Oncol 2018; 19: 207-215.
42. Horniblow R D, Bedford M, Hollingworth R, Evans S, Sutton E, Lal N et al. BRAF mutations are associated with increased iron regulatory protein-2 expression in colorectal tumoigenesis. Cancer Sci 2017; 108: 1135-1143.
43. Tacchini L, Bianchi L, Bemelli-Zazzera A, Cairo G. Transferrin Receptor Induction by Hypoxia: HIF-1-MEDIATED TRANSCRIPTIONAL ACTIVATION AND CELL-SPECIFIC POST-TRANSCRIPTIONAL REGULATION. J Biol Chem 1999; 274: 24142-24146.
44. Lok C N, Ponka P. Identification of a Hypoxia Response Element in the Transferrin Receptor Gene. J Biol Chem 1999; 274: 24147-24152.
45. Bayeva M, Khechaduri A, Puig S, Chang H-C, Patial S, Blackshear P J et al. mTOR regulates cellular iron homeostasis through tristetraprolin. Cell Metab 2012; 16: 645-657.
46. Nick McElhinny, S. A., et al., Division of labor at the eukaryotic replication fork. Mol Cell, 2008. 30(2): p. 137-44.
47. 35. Pursell, Z. F., et al., Yeast DNA polymerase epsilon participates in leading-strand DNA replication. Science, 2007. 317(5834): p. 127-30.
48. 36. Paul, V. D. and R. Lill, Biogenesis of cytosolic and nuclear iron-sulfur proteins and their role in genome stability. Biochim Biophys Acta, 2015. 1853(6): p. 1528-39. 49.37. Jozwiakowski, S. K., S. Kummer, and K. Gari, Human DNA polymerase delta requires an iron-sulfur duster for high-fidelity DNA synthesis. Life Science Alliance, 2019. 2(4).
50. Qin, Q., et al., Elevated expression of POLD1 is associated with poor prognosis in breast cancer. Oncol Left, 2018. 16(5): p. 5591-5598.
51. Buchanan, D. D., et al., Risk of colorectal cancer for carriers of a germ-line mutation in POLE or POLD1. Genet Med, 2018. 20(8): p. 890-895.
52. Xu, Y., et al., SIRT1 promotes proliferation, migration, and invasion of breast cancer cell line MCF-7 by upregulating DNA polymerase delta1 (POLD1). Biochem Biophys Res Commun, 2018. 502(3): p. 351-357.
53. Torti S V, Manz D H, Paul B T, Blanchette-Farra N, Torti F M. Iron and Cancer. Annu Rev Nutr 2018; 38: 97-125.
54. Bergeron R J, Wiegand J, Brittenham G M. HBED ligand: preclinical studies of a potential alternative to deferoxamine for treatment of chronic iron overload and acute iron poisoning. Blood 2002; 99: 3019-3026.
55. Neufeld E J. Oral chelators deferasirox and deferiprone for transfusional iron overload in thalassemia major new data, new questions. Blood. 2006; 107:3436-41.
56. Yamasaki T, Terai S, Sakaida I. Deferoxamine for Advanced Hepatocellular Carcinoma. N Engl J Med 2011; 365: 576-578.
57. Olivieri N F, Brittenham G M. Iron-Chelating Therapy and the Treatment of Thalassemia. Blood 1997; 89: 739-761.
58. Cappellini M D, Pattoneri P. Oral Iron Chelators. Annu Rev Med 2009; 60: 25-38.
59. Kicic A, Chua A C G, Baker E. Effect of iron chelators on proliferation and iron uptake in hepatoma cells. Cancer 2001; 92: 3093-3110.
60. Ohara T, Noma K, Urano S, Watanabe S, Nishitani S, Tomono Y et al. A novel synergistic effect of iron depletion on antiangiogenic cancer therapy. Int J Cancer 2013; 132: 2705-2713.
61. Cheng G, Zielonka J, Hardy M, Ouar O, Chitambar C R, Dwinell M B et al. Synergistic inhibition of tumor cell proliferation by metformin and mito-metformin in the presence of iron chelators. Oncotarget 2019; 10: 3518-3532.
62. Tury S, Assayag F, Bonin F, Chateau-Joubert S, Servely J-L, Vacher S et a. The iron chelator deferasirox synergises with chemotherapy to treat triple-negative breast cancers. J Pathol 2018; 246: 103-114.
63. Shinoda S, Kaino S, Amano S, Harima H, Matsumoto T, Fujisawa K et al. Deferasirox, an oral iron chelator, with gemcitabine synergistically inhibits pancreatic cancer cell growth in vitro and in vivo. Oncotarget 2018; 9: 28434-28444.
64. Dent P. Investigational CHK1 inhibitors in early phase clinical trials for the treatment of cancer. Expert Opin Investig Drugs 2019; 28: 1095-1100.
65. Jia J, Claude-Taupin A, Gu Y, Choi S W, Peters R, Bissa B et al. Galectin-3 Coordinates a Cellular System for Lysosomal Repair and Removal. Dev Cell 2020; 52: 69-87.e8.

### References Second Set

1. Siegel R L, Miller K D, Fuchs H E, et al. Cancer Statistics, 2021. CA Cancer J Clin 2021; 71:7-33.
2. Soerjomataram I, Bray F. Planning for tomorrow: global cancer incidence and the role of prevention 2020-2070. Nat Rev Clin Oncol 2021; 18:663-672.
3. Cross A J, Ferrucci L M, Risch A, et al. A large prospective study of meat consumption and colorectal cancer risk: an investigation of potential mechanisms underlying this association. Cancer Res 2010; 70:2406-2414.
4. Oates P-S, West A-R. Heme in intestinal epithelial cell turnover, differentiation, detoxification, inflammation, carcinogenesis, absorption and motility. World J Gastroenterol 2006; 12:4281-4295.
5. Bastide N M, Chenni F, Audebert M, et al. A Central Role for Heme Iron in Colon Carcinogenesis Associated with Red Meat Intake. Cancer Res 2015:75:870-879.
6. Xue X, Shah Y M. Intestinal iron homeostasis and colon tumorigenesis. Nutrients 2013; 5:2333-2351.
7. Xue X, Taylor M, Anderson E, et al. Hypoxia-inducible factor-2a activation promotes colorectal cancer progression by dysregulating iron homeostasis. Cancer Res 2012; 72:2285-2293.
8. Xue X, Ramakrishnan S K, Weisz K, et al. Iron Uptake via DMT1 Integrates Cell Cycle with JAK-STAT3 Signaling to Promote Colorectal Tumorigenesis. Cell Metab 2016; 24:447-461.
9. Schwartz A J, Goyert J W, Solanki S, et al. Hepcidin sequesters iron to sustain nucleotide metabolism and mitochondrial function in colorectal cancer epithelial cells. Nat Metab 2021; 3:969-982.
10. Hentze M W, Muckenthaler M U, Andrews N C. Balancing Acts: Molecular Control of Mammalian Iron Metabolism. Cell 2004; 117:285-297.
11. Parmley R T, Barton J C, Conrad M E. Ultrastructural localization of transferrin, transferrin receptor, and iron-binding sites on human placental and duodenal microvilli. Br J Haematol 1985; 60:81-89.
12. Anderson G J, Powell L W, Halliday J W. Transferrin receptor distribution and regulation in the rat small intestine: Effect of iron stores and erythropoiesis. Gastroenterology 1990; 98:576-585.
13. Banerjee D, Flanagan P R, Cluett J, et al. Transferrin receptors in the human gastrointestinal tract: Relationship to body iron stores. Gastroenterology 1986; 91:861-869.
14. Morales M, Xue X. Targeting iron metabolism in cancer therapy. Theranostics 2021; 11:8412-8429.
15. Huang S M A, Mishina Y M, Liu S, et al. Tankyrase inhibition stabilizes axin and antagonizes Wnt signalling. Nature 2009; 461:614-620.
16. Uhlén M, Fagerberg L, Hallström B M, et al. Tissue-based map of the human proteome. Science 2015; 347:lssue 6220.
17. Chandrashekar D S, Bashel B, Balasubramanya S A H, et al. UALCAN: A Portal for Facilitating Tumor Subgroup Gene Expression and Survival Analyses. Neoplasia 2017; 19:649-58.
18. Goss K H, Groden J. Biology of the adenomatous polyposis coli tumor suppressor. J Clin Oncol 2000; 18(9):1967-79.
19. Watson A, Lipina C, McArdle H J, at al. Iron depletion suppresses mTORC1-directed signalling in intestinal Caco-2 cells via induction of REDD1. Cell Signal 2016; 28:412-424.
20. Faller W J, Jackson T J, Knight J R, et al. mTORC1-mediated translational elongation limits intestinal tumour initiation and growth. Nature 2015; 517:497-500.
21. Huang D W, Sherman B T, Tan Q, et al. DAVID Bioinformatics Resources: expanded annotation database and novel algorithms to better extract biology from large gene lists. Nucleic Acids Res 2007; 35:169-175.
22. Palles C, Cazier, J B, Howarth K M, et al. Germline mutations affecting the proofreading domains of POLE and POLD1 predispose to colorectal adenomas and carcinomas. Nat. Genet 2013; 45:136-144.
23. Wang G L, Semenza G L. Desferrioxamine induces erythropoietin gene expression and hypoxia-inducible factor 1 DNA-binding activity: implications for models of hypoxia signal transduction. Blood 1993; 82:3610-3615.
24. U, B. and M. Y. Lee. Transcriptional regulation of the human DNA polymerase delta catalytic subunit gene POLD1 by p53 tumor suppressor and Sp1. J Biol Chem, 2001; 276:29729-39.
25. Gao S, Song Q, Liu J, et al. E2F1 mediates the downregulation of POLD1 in replicative senescence. Cell Mol Life Sci 2019; 76:2833-2850.
26. Leung J Y, Ehmann G L, Giangrande P H, et al. A role for Myc in facilitating transcription activation by E2F1. Oncogene 2008:27:4172-4179.
27. Kyriakou D, Eliopoulos A G, Papadakis A, et al. Decreased expression of c-myc oncoprotein by peripheral blood mononuclear cells in thalassaemia patients receiving desferrioxamine. Eur J Haematol 1998; 60:21-27.
28. He T C, Sparks A B, Rago C, et al. Identification of c-MYC as a target of the APC pathway. Science 1998; 281:1509-12.
29. Siyuan S, Tania C, Stephen P, et al. Wnt inhibitor screen reveals iron dependence of beta-catenin signaling in cancers. Cancer Res 2011; 71:7628-39.
30. Radulescu S, Brookes M J, Salgueiro P, et al. Luminal iron levels govern intestinal tumorigenesis after Apc loss in vivo. Cell Rep 2012; 2:270-282.
31. Nurtjahja T E, Fu D, Phang J M, et al. Iron chelation regulates cyclin D1 expression via the proteasome: a link to iron deficiency-mediated growth suppression. Blood 2006; 109:4045-54.
32. Jho E H, Zhang T, Domon C, et al. Wnt/beta-catenin/Tcf signaling induces the transcription of Axin2, a negative regulator of the signaling pathway. Mol Cell Biol 2002; 22:1172-83.
33. Haikarainen T, Krauss S, Lehtio L. Tankyrases: structure, function and therapeutic implications in cancer. Curr Pharm Des 2014; 20:6472-88.
34. Conde C D, Petronczkl O Y, Barls S, et al. Polymerase delta deficiency causes syndromic immunodeficiency with replicative stress. J Clin Invest 2019; 129:4194-4206.
35. Zou L, Elledge S J. Sensing DNA damage through ATRIP recognition of RPA-ssDNA complexes. Science 2003; 300:1542-8.
36. Zhao H, Piwnica-Worms H. ATR-mediated checkpoint pathways regulate phosphorylation and activation of human Chk1. Mol Cell Biol 2001; 21:4129-39.
37. Furuta T, Takemura H, Liao Z Y, et al. Phosphorylation of histone H2AX and activation of Mre11, Rad50, and Nbs1 in response to replication-dependent DNA double-strand breaks induced by mammalian DNA topoisomerase I cleavage complexes. J Biol Chem 2003:278:20303-12.
38. Hocke S, Guo Y, Job A, et al. A synthetic lethal screen identifies ATR-inhibition as a novel therapeutic approach for POLD1-deficient cancers. Oncotarget 2016; 7:7080-7095.
39. Rogers R F, Walton M I, Cherry D L, et al. CHK1 Inhibition Is Synthetically Lethal with Loss of B-Family DNA Polymerase Function in Human Lung and Colorectal Cancer Cells. Cancer Res 2020; 80:1735-1747.
40. Lee J M, Nair J, Zimmer A, et al. Prexasertib, a cell cycle checkpoint kinase 1 and 2 inhibitor, in BRCA wild-type recurrent high-grade serous ovarian cancer: a first-in-class proof-of-concept phase 2 study. Lancet Oncol 2018; 19:207-215.
41. Daud Al, Ashworth M T, Strosberg J, et al., Phase I dose-escalation trial of checkpoint kinase 1 inhibitor MK-8776 as monotherapy and in combination with gemcitabine in patients with advanced solid tumors. J Clin Oncol 2015; 33:1060-6.
42. Sansom O J, Reed K R, Hayes A J, et al. Loss of Apc in vivo immediately perturbs Wnt signaling, differentiation, and migration. Genes Dev 2004; 18:1385-90.
43. Feng Y, Sakamoto N, Wu R, et al. Tissue-Specific Effects of Reduced s-catenin Expression on Adenomatous Polyposis Coli Mutation-Instigated Tumorigenesis in Mouse Colon and Ovarian Epithelium. PLoS Genet 2015; 11: e1005638
44. Nicolas E, Golemis E A, Arora S. POLD1: Central mediator of DNA replication and repair, and implication in cancer and other pathologies. Gene 2016; 590-128-141.
45. Yamasaki T, Terai S, Sakaida I. Deferoxamine for Advanced Hepatocellular Carcinoma. N Engl J Med 2011; 365:576-578.
46. Olivieri N F, Brittenham G M. Iron-Chelating Therapy and the Treatment of Thalassemia. Blood 1997; 89:739-761.
47. Cappellini M D, Pattoneri P. Oral Iron Chelators. Annu Rev Med 2009; 60:25-38.
48. Kicic A, Chua A C G, Baker E. Effect of iron chelators on proliferation and iron uptake in hepatoma cells. Cancer 2001; 92:3093-3110.
49. Neufeld E J. Oral chelators deferasirox and deferiprone for transfusional iron overload in thalassemia major new data, new questions. Blood 2006:107:3436-41.
50. Turner J, Koumenis C, Kute T E, et al. Tachpyridine, a metal chelator, induces G2 cell-cycle arrest, activates checkpoint kinases, and sensitizes cells to ionizing radiation. Blood 2005; 106:3191-9.
51. Yap T A, Plummer R, Azad N S, et al. The DNA Damaging Revolution: PARP Inhibitors and Beyond. Am Soc Clin Oncol Educ B 2019; 39:185-95.
52. Dent P. Investigational CHK1 inhibitors in early phase clinical trials for the treatment of cancer. Expert Opin Investig Drugs 2019:28:1095-1100.

1. A method of treating cancer in a patient or subject in need thereof comprising administering to said patient or subject an anti-cancer effective amount of a composition comprising at least one an iron chelator compound in combination with an anticancer agent or therapy, wherein said anticancer agent is a checkpoint 1 (Chk1) inhibitor, a ATR inhibitor, a DNA damaging agent, radiotherapy or a mixture thereof.

2. The method according to claim 1 wherein said iron chelator compound is selected from the group consisting of deferoxamine, deferasirox, Dp44mT, dexrazoxane, ciclopirox, dexrazoxane HCl (ICRF-187), pentetate calcium trisodium hydrate, 2,3-dihydroxybenzoic acid, VLX600, L-mimosine, N-NE3TA-NCS, CAB-NE3TA, DFT (2-(3′-hydroxypyrid-2′-yl)4-methyl-delta2-thiazoline-4(S)-carboxylic acid; desferrithiocin), 4-(OH)-DADFT, 4′-(HO)-DADMDFT ((S)-2-(2,4-dihydroxyphenyl)-4,5-dihydro-4-thiazolecarboxylic acid), BDU ((S,S)-1,11-bis[5-(4-carboxy-4,5-dihydrothiazol-2-yl)-2,4-dihydroxyphenyl]-4,8-dioxaundecane), ICL6770A (4-[3,5-bis-(hydroxyphenyl)-1,2,4-triazol-1-yl]-benzoic acid), DFP (3-Hydroxy-1,2-dimethyl-4(1H)-pyridone; Deferiprone), CP94 (Diethyl hydroxypyridinone), CP502 (1,6-dimethyl-3-hydroxy-4-(1H)-pyridinone-2-carboxy-(N-methyl)-amide hydrochloride), TREN-(Me-3,2-HOPO) (N,N′,N″-tris[(3-hydroxy-1-methyl-2-oxo-1,2-didehydropyrid-4-yl)carboxamidoethyl]amine), Pr-(Me-3,2-HOPO) (3-Hydroxy-1-methyl-2-oxo-1,2-dihydro-pyridine-4-carboxylic acid propylamide), Tachpyridine (N,N′,N″-tris(2-pyridylmethyl)-cis,cis-1,3,5-triaminocyclohexane),PIH, SIH (Salicylaldehyde isonicotinoyl hydrazone), 311 (2-hydroxy-1-naphthylaldehyde isonicotinoyl hydrazone), 5-HP (5-hydroxypicolinaldehyde thiosemicarbazone), D-Exo 772SM, PCIH, INH, Deferitazole, EDTA, DTPA, Succimer, Trientine, BPS, PCTH, PCBH, PCBBH, PCAH, PCHH, FIH, Quercetin, or a pharmaceutically acceptable salt or mixture thereof.

3. The method according to claim 1 wherein said iron chelator compound is selected from the group consisting of deferoxamine, deferasirox, eferiprone, Dp44mT, dexrazoxane, ciclopirox, dexrazoxane HCl (ICRF-187), pentetate calcium trisodium hydrate, 2,3-dihydroxybenzoic acid, VLX600, L-mimosine, triapine (3-AP), N-NE3TA-NCS, CAB-NE3TA or a pharmaceutically acceptable salt or mixture thereof.

4. (canceled)

5. The method according to claim 1 wherein said Chk1 inhibitor is a compound selected from the group consisting of prexasertib, UCN-01, SRA737, AZD7762, rabusertib (LY2603618), MK-8776 (SCH 900776), CHIR-124, PF-477736, VX-803 (M4344), GDC-0575 (ARRY-575), PD0166285, CCT245737 (SRA737, PNT-737), SAR-020106, SB 218078, prexasertib, UCN-01, SRA737, AZD7762, rabusertib (LY2603618), MK-8776 (SCH 900776), CHIR-124, PF-477736, VX-803 (M4344), GDC-0575 (ARRY-575), PD0166285, SAR-020106, SB 218078, TCS2312, aminothiadiazole and aminothiadiazole conjugated cyanopyridines, CCT245737 (SRA737, PNT-737), CCT245737(S), CCT244747, GDC-0425, GNE-783, GNE-900, GO-6976, MU-380, NSC30049, PD0407824, PD-321852, V158411 or a pharmaceutically acceptable salt or mixture thereof.

6. (canceled)

7. The method according to claim 1 wherein said iron chelator compound is desferoxamine, deferasirox or a mixture thereof.

8. (canceled)

9. (canceled)

10. The method according to claim 1 wherein said ATR inhibitor is VE-822 (VX-970, M6620), Elimusertib (BAY1895344), VX-803, EPT-46464, AZ20, ceralasertib (AZD 6738) and VE-821, CGK 733, schisandrin B, HAMNO, Torin 2 or a mixture thereof.

11. (canceled)

12. (canceled)

13. The method according to claim 1 wherein said DNA damaging agent is an antimetabolite, a topoisomerase inhibitor or an anthracycline antibiotic or a mixture thereof.

14. The method according to claim 1 wherein said DNA damaging agent is melphalan, cyclophosphamide, temozolomide, carmustine (BCNU), fotemustine, ecteinascidin-743, duocarmycin A, dacarbazine, laromustine, duocarmycin A, mithramycin A, CC-1065, adozelesin, carzelesin, bizelesin, tallimustine, diflometecan, mchlorethamine, chlorambucil, bendamustine, spiromustine, uramustine, estramustine phosphate, DHEA mustard, prechimustine, imidazole mustard, ifosfamide, trofosfamide, mafosfamide, sarCNU, iomustine (CCNU), lomustine, semustine, ranimustine, nimustine, streptozotocin, chlorozotocin, 1,2-bis(sulfonyl)hydrazine, procarbazine, p-methylbenzylhydrazine, thiotepa, triethylene melamine, hexamethylmelamine, pentamethylmelamine, triaziquone, arbazilquinone, AZQ (diaziquinone), BZQ, DZQ, busulfan, dimethylbusulfan, treosulfan, hepsulfan, mannosulfan, Methylene dimethane sulfonate, illudin A, illudin B, illudin M, illudin S, HMAF, cisplatin, carboplatin, nedaplatin, iproplatin, oxaloplatin, lobaplatin, strataplatin, spiroplatin, picoplatin, heptaplatin, triplatin tetranitrate, Indicine-N-oxide, Dianhydrogalactitol, Diacetyl dianhydrogalactitol, teroxirone, ducarmycin-SA, KW 2189, distamycin, tallimustin, MEN 10710, MEN 10716, brostalicin, PNU-151807, mithramycin A, mithramycin SK, chromomycin A, chromocyclomycin, olivomycin, UCH9, mitomycin, streptonigrin, porfirmycin, KW-2149, daunorubin, doxorubin, idarubin, amrubicin, epirubicin, valrubicin, pirarubicin, berubicin, carubicin, esorubicin, detorubicin, duborimycin, zorubicin, aclacinomycin, marcellomycin, musettamycin, ametantrone, mitoxantrone, pixantrone, teloxantrone, piroxantrone, losoxantrone, bisantrene, calicheamicin, esperamicin, namenamicin, shishjimicin, neocarinostatin, lidamycin, kedarcidin, maduropeptin, N1999A2, actinomycin D, nleomycin, fostriecin, irinotecan, rubitecan, exatecan, kareniticin, topotecan, lurtotecan, CKD602, camptothecin, gimatecan, diflomatecan, rebeccamycin, NB-506, edotecarin, AT2433, lamellarin A, lamellarin C, lamellarin D, lamellarin G, lamellarin L, lamellarin M, NSC 314622, NSC 706744, NSC 726972, NSC 725766, NSC 724988, NSC 727357, etoposide, teniposide, chloroquinoxoline sulfonamide, XK469, asulacrine, razoxane, elliptinium, amonafide, batracylin, aminopterin, methotrexate, pralatrexate, edatrexate, talotrexin, pemetrexed, ralitrexed, nolatrexted, OSI-7904L, thymectacin, lomatrexole, dapsone, AG2034, triazinate, trifluridine, 5-Fluorouracil, 5-iododeoxyuridine, 5-bromodeoxyuridine, 6-azaurdine, cytarabine, fazarabine, decitabine, gemcitibine, sapacitabine, penclomedine, m-azidopyramethamine, metoprine, hydroxyurea, 6-mercaptopurine, 6-thioguanine, azathioprine, methylthioinosine, thioguanine deoxyriboside, cladribine, vidarabine, 2′-deoxycoformicin, fludarabine, clofarabine, nelarabine, L-alanosine, forodesine, inosine dialdehyde, tiazofurin, triapine or a mixture thereof.

15. (canceled)

16. The method according to claim 1 wherein said radiotherapy is external beam radiation therapy, contact x-ray brachytherapy, brachytherapy (sealed source radiotherapy), radionuclide therapy and/or intraoperative radiotherapy.

17. The method according to claim 1 further comprising administering to said patient an additional anti-cancer agent which is other than a DNA damaging agent.

18. (canceled)

19. The method according to claim 1 wherein said cancer is squamous-cell carcinoma, basal cell carcinoma adenocarcinoma, hepatocellular carcinoma, renal cell carcinoma, leukemia, Burkitt's lymphoma, Non-Hodgkin's lymphoma, benign and malignant melanoma, myeloproliferative disease, Ewing's sarcoma, hemangiosarcoma, Kaposi's sarcoma, liposarcoma, myosarcomas, peripheral neuroepithelioma, glioma, astrocytoma, oligodendroglioma, ependymomas, gliobastomas, neuroblastomas, ganglioneuromas, gangliogliomas, medulloblastomas, a pineal cell tumor, meningioma, meningeal sarcoma, neurofibroma, Schwannoma; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, rectal cancer carcinosarcoma, Hodgkin's disease; Wilms' tumor or teratocarcinoma.

20. The method according to claim 1 wherein said cancer is breast, bladder, cervical, colon, head and neck, Hodgkin lymphoma, liver, lung, renal cell, skin, stomach or rectal cancer.

21. (canceled)

22. (canceled)

23. A pharmaceutical composition comprising an anti-cancer effective amount of an iron chelator compound in combination with at least one agent selected from the group consisting of a checkpoint kinase 1 (Chk1) inhibitor, an ATR inhibitor, a DNA damaging agent and mixtures thereof further in combination with a pharmaceutically acceptable carrier, additive or excipient.

24. The composition according to claim 23 wherein said iron chelator compound is selected from the group consisting of deferoxamine, deferasirox, Dp44mT, dexrazoxane, ciclopirox, dexrazoxane HCl (ICRF-187), pentetate calcium trisodium hydrate, 2,3-dihydroxybenzoic acid, VLX600, L-mimosine, N-NE3TA-NCS, CAB-NE3TA, DFT (2-(3′-hydroxypyrid-2′-yl)4-methyl-delta2-thiazoline-4(S)-carboxylic acid; desferrithiocin), 4-(OH)-DADFT, 4′-(HO)-DADMDFT ((S)-2-(2,4-dihydroxyphenyl)-4,5-dihydro-4-thiazolecarboxylic acid), BDU ((S,S)-1,11-bis[5-(4-carboxy-4,5-dihydrothiazol-2-yl)-2,4-dihydroxyphenyl]-4,8-dioxaundecane), ICL6770A (4-[3,5-bis-(hydroxyphenyl)-1,2,4-triazol-1-yl]-benzoic acid), DFP (3-Hydroxy-1,2-dimethyl-4(1H)-pyridone; Deferiprone), CP94 (Diethyl hydroxypyridinone), CP502 (1,6-dimethyl-3-hydroxy-4-(1H)-pyridinone-2-carboxy-(N-methyl)-amide hydrochloride), TREN-(Me-3,2-HOPO) (N,N′,N″-tris[(3-hydroxy-1-methyl-2-oxo-1,2-didehydropyrid-4-yl)carboxamidoethyl]amine), Pr-(Me-3,2-HOPO) (3-Hydroxy-1-methyl-2-oxo-1,2-dihydro-pyridine-4-carboxylic acid propylamide), Tachpyridine (N,N′,N″-tris(2-pyridylmethyl)-cis,cis-1,3,5-triaminocyclohexane),PIH, SIH (Salicylaldehyde isonicotinoyl hydrazone), 311 (2-hydroxy-1-naphthylaldehyde isonicotinoyl hydrazone), 5-HP (5-hydroxypicolinaldehyde thiosemicarbazone), D-Exo 772SM, PCIH, INH, Deferitazole, EDTA, DTPA, Succimer, Trientine, BPS, PCTH, PCBH, PCBBH, PCAH, PCHH, FIH, Quercetin, or a pharmaceutically acceptable salt or mixture thereof.

25. (canceled)

26. (canceled)

27. The composition according to claim 23 wherein said Chk1 inhibitor is prexasertib, UCN-01, SRA737, AZD7762, rabusertib (LY2603618), MK-8776 (SCH 900776), CHIR-124, PF-477736, VX-803 (M4344), GDC-0575 (ARRY-575), PD0166285, CCT245737 (SRA737, PNT-737), SAR-020106, SB 218078, prexasertib, UCN-01, SRA737, AZD7762, rabusertib (LY2603618), MK-8776 (SCH 900776), CHIR-124, PF-477736, VX-803 (M4344), GDC-0575 (ARRY-575), PD0166285, SAR-020106, SB 218078, TCS2312, aminothiadiazole and aminothiadiazole conjugated cyanopyridines, CCT245737 (SRA737, PNT-737), CCT245737(S), CCT244747, GDC-0425, GNE-783, GNE-900, GO-6976, MU-380, NSC30049, PD0407824, PD-321852, V158411 or a pharmaceutically acceptable salt or mixture thereof.

28. (canceled)

29. (canceled)

30. (canceled)

31. (canceled)

32. The composition according to claim 23 wherein said ATR inhibitor is VE-822 (VX-970, M6620), Elimusertib (BAY1895344), VX-803, EPT-46464, AZ20, ceralasertib (AZD 6738) and VE-821, CGK 733, schisandrin B, HAMNO, Torin 2 or a mixture thereof.

33. (canceled)

34. (canceled)

35. The composition according to claim 34 wherein said DNA damaging agent is melphalan, cyclophosphamide, temozolomide, carmustine (BCNU), fotemustine, ecteinascidin-743, duocarmycin A, dacarbazine, laromustine, duocarmycin A, mithramycin A, CC-1065, adozelesin, carzelesin, bizelesin, tallimustine, diflometecan, mechlorethamine, chlorambucil, bendamustine, spiromustine, uramustine, estramustine phosphate, DHEA mustard, prechimustine, imidazole mustard, ifosfamide, trofosfamide, mafosfamide, sarCNU, iomustine (CCNU), lomustine, semustine, ranimustine, nimustine, streptozotocin, chlorozotocin, 1,2-bis(sulfonyl)hydrazine, procarbazine, p-methylbenzylhydrazine, thiotepa, triethylene melamine, hexamethylmelamine, pentamethylmelamine, triaziquone, arbazilquinone, AZQ (diaziquinone), BZQ, DZQ, busulfan, dimethylbusulfan, treosulfan, hepsulfan, mannosulfan, Methylene dimethane sulfonate, illudin A, illudin B, illudin M, illudin S, HMAF, cisplatin, carboplatin, nedaplatin, iproplatin, oxaloplatin, lobaplatin, strataplatin, spiroplatin, picoplatin, heptaplatin, triplatin tetranitrate, Indicine-N-oxide, Dianhydrogalactitol, Diacetyl dianhydrogalactitol, teroxirone, ducarmycin-SA, KW 2189, distamycin, tallimustin, MEN 10710, MEN 10716, brostalicin, PNU-151807, mithramycin A, mithramycin SK, chromomycin A, chromocyclomycin, olivomycin, UCH9, mitomycin, streptonigrin, porfirmycin, KW-2149, daunorubin, doxorubin, idarubin, amrubicin, epirubicin, valrubicin, pirarubicin, berubicin, carubicin, esorubicin, detorubicin, duborimycin, zorubicin, aclacinomycin, marcellomycin, musettamycin, ametantrone, mitoxantrone, pixantrone, teloxantrone, piroxantrone, losoxantrone, bisantrene, calicheamicin, esperamicin, namenamicin, shishjimicin, neocarinostatin, lidamycin, kedarcidin, maduropeptin, N1999A2, actinomycin D, nleomycin, fostriecin, irinotecan, rubitecan, exatecan, kareniticin, topotecan, lurtotecan, CKD602, camptothecin, gimatecan, diflomatecan, rebeccamycin, NB-506, edotecarin, AT2433, lamellarin A, lamellarin C, lamellarin D, lamellarin G, lamellarin L, lamellarin M, NSC 314622, NSC 706744, NSC 726972, NSC 725766, NSC 724988, NSC 727357, etoposide, teniposide, chloroquinoxoline sulfonamide, XK469, asulacrine, razoxane, elliptinium, amonafide, batracylin, aminopterin, methotrexate, pralatrexate, edatrexate, talotrexin, pemetrexed, ralitrexed, nolatrexted, OSI-7904L, thymectacin, lomatrexole, dapsone, AG2034, triazinate, trifluridine, 5-Fluorouracil, 5-iododeoxyuridine, 5-bromodeoxyuridine, 6-azaurdine, cytarabine, fazarabine, decitabine, gemcitibine, sapacitabine, penclomedine, m-azidopyramethamine, metoprine, hydroxyurea, 6-mercaptopurine, 6-thioguanine, azathioprine, methylthioinosine, thioguanine deoxyriboside, cladribine, vidarabine, 2′-deoxycoformicin, fludarabine, clofarabine, nelarabine, L-alanosine, forodesine, inosine dialdehyde, tiazofurin, triapine or a mixture thereof.

36. The composition according to claim 23 formulated to treat a cancer wherein said cancer is squamous-cell carcinoma, basal cell carcinoma adenocarcinoma, hepatocellular carcinoma, renal cell carcinoma, leukemia, Burkitt's lymphoma, Non-Hodgkin's lymphoma, benign and malignant melanoma, myeloproliferative disease, Ewing's sarcoma, hemangiosarcoma, Kaposi's sarcoma, liposarcoma, myosarcomas, peripheral neuroepithelioma, glioma, astrocytoma, oligodendroglioma, ependymomas, gliobastomas, neuroblastomas, ganglioneuromas, gangliogliomas, medulloblastomas, a pineal cell tumor, meningioma, meningeal sarcoma, neurofibroma, Schwannoma; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, rectal cancer carcinosarcoma, Hodgkin's disease; Wilms' tumor or teratocarcinoma.

37. (canceled)

38. (canceled)

39. The composition according to claim 23 in oral or parenteral dosage form.

40. (canceled)

41. (canceled)
